(19) United States
(12) Patent Application Publication
(10) Pub. No.: US 2026/0007381 A1
(43) Pub. Date: Jan. 8, 2026

(54) MEDICAL INFORMATION PROCESSING METHOD, MEDICAL INFORMATION PROCESSING DEVICE, AND STORAGE MEDIUM

(71) Applicant: CANON MEDICAL SYSTEMS CORPORATION, Otawara-shi (JP)

(72) Inventors: Masakazu MATSUURA, Nasushiobara (JP); Takuya NEMOTO, Otawara (JP); Hiroaki KIJI, Utsunomiya (JP); Yojiro SUZUKI, Oyama (JP); Naruomi AKINO, Ota (JP)

(73) Assignee: CANON MEDICAL SYSTEMS CORPORATION, Otawara-shi (JP)

(21) Appl. No.: 19/254,135

(22) Filed: Jun. 30, 2025

(30) Foreign Application Priority Data
Jul. 3, 2024 (JP) 2024-107329

(51) Int. Cl. A61B 6/00; A61B 6/46

(52) U.S. Cl. CPC A61B 6/5258; A61B 6/461; A61B 6/5205

(57) ABSTRACT

According to an embodiment, a medical information processing method includes acquiring projection data of X-rays radiated to a subject, generating a medical image from the projection data by a reconstruction process, separating the medical image into a signal image and a noise image by a whitening transform, denoising at least one of the signal image and the noise image using a first trained model, generating a denoised medical image by coupling both images that have been denoised by an inverse whitening transform, and generating the denoised medical image by coupling one image that has been denoised between the signal image and the noise image and the other image that has not been denoised.

### CROSS-REFERENCE TO RELATED APPLICATION

Priority is claimed on Japanese Patent Application No. 2024-107329, filed Jul. 3, 2024, the content of which is incorporated herein by reference.

### FIELD

Embodiments disclosed in the present specification and the drawings relate to a medical information processing method, a medical information processing device, and a storage medium.

### BACKGROUND

In recent years, noise reduction technology using deep learning has been actively proposed for X-ray computed tomography (CT) devices. As a result, the noise reduction technology has contributed greatly to the reduction of radiation exposure. Moreover, in spectral imaging of dual-energy CT (DECT) and photon-counting CT (PCCT), it is always necessary to consider the reduction of radiation exposure, and noise reduction is necessary technology.

In recent years, noise reduction methods using deep learning in spectral imaging have also been proposed. However, there is still room for improvement in the accuracy of noise reduction in conventional technology.

### BRIEF DESCRIPTION OF THE DRAWINGS

FIG. 1 is a diagram showing an example of a configuration of a medical information processing system in a first embodiment.

FIG. 2 is a diagram showing an example of a configuration of an X-ray CT device in the first embodiment.

FIG. 3 is a flowchart showing an example of a flow of a series of processing steps of the X-ray CT device in the first embodiment.

FIG. 4 is a diagram schematically showing a flow of a series of processing steps of the X-ray CT device in the first embodiment.

FIG. 5 is a diagram showing an example of a configuration of a learning device in the first embodiment.

FIG. 6 is a flowchart showing a flow of a series of processing steps of the learning device according to the first embodiment.

FIG. 7 is a diagram schematically showing a flow of a series of processing steps of the learning device according to the first embodiment.

FIG. 8 is a diagram showing an example of a configuration of a DAS of an X-ray CT device according to a second embodiment.

FIG. 9 is a diagram showing an example of a functional configuration of a reconstruction function according to the second embodiment.

FIG. 10 is a diagram schematically showing a flow of a series of processing steps of an X-ray CT device in a third embodiment.

FIG. 11 is a diagram schematically showing a flow of a series of processing steps of a learning device according to the third embodiment.

FIG. 12 is a diagram schematically showing a flow of a series of processing steps of an X-ray CT device in a fourth embodiment.

FIG. 13 is a diagram schematically showing a flow of a series of processing steps of a learning device according to the fourth embodiment.

FIG. 14 is a flowchart showing an example of a flow of a series of processing steps of an X-ray CT device in a fifth embodiment.

FIG. 15 is a diagram schematically showing a flow of a series of processing steps of the X-ray CT device according to the fifth embodiment.

FIG. 16 is a flowchart showing a flow of a series of processing steps of a learning device according to the fifth embodiment.

FIG. 17 is a diagram schematically showing a flow of a series of processing steps of the learning device according to the fifth embodiment.

FIG. 18 is a diagram schematically showing a flow of a series of processing steps of a learning device according to a sixth embodiment.

FIG. 19 is a diagram schematically showing a flow of a series of processing steps of a learning device according to a seventh embodiment.

### DETAILED DESCRIPTION

Hereinafter, a medical information processing method, a medical information processing device, and a storage medium according to embodiments will be described with reference to the drawings.

According to an embodiment, there is provided a medical information processing method including: acquiring projection data of X-rays radiated to a subject; generating a medical image from the projection data by a reconstruction process; separating the medical image into a signal image in which the number of signal components is more than the number of noise components and a noise image in which the number of noise components is more than the number of signal components by a whitening transform; denoising at least one of the signal image and the noise image using a first trained model that is a machine learning model trained on the basis of a training dataset in which a first output image having less noise than a first input image is associated as a target with the first input image; generating a denoised medical image that is the medical image that has been denoised by coupling the signal image that has been denoised and the noise image that has been denoised by an inverse whitening transform when both the signal image and the noise image have been denoised; and generating the denoised medical image by coupling one image that has been denoised between the signal image and the noise image and the other image that has not been denoised by the inverse whitening transform when one of the signal image and the noise image has been denoised. According to this process, it is possible to improve the accuracy of noise reduction in spectral imaging.

### First Embodiment

### [Configuration of Medical Information Processing System]

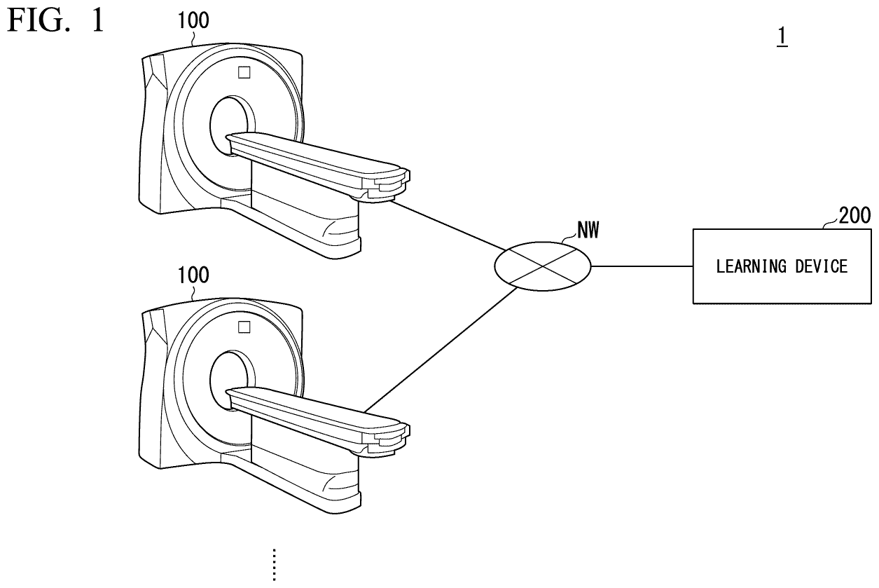

FIG. 1 is a diagram illustrating an example of a configuration of a medical information processing system 1 in a first embodiment. The medical information processing system 1 includes, for example, a plurality of medical image diagnosis devices 100 and a learning device 200. The medical image diagnosis devices 100 and the learning device 200 are communicatively connected via a communication network NW.

The communication network NW may be a general information communication network using telecommunication technologies. For example, the communication network NW includes a telephone communication line network, an optical fiber communication network, a cable communication network, a satellite communication network, and the like as well as a wireless/wired local area network (LAN) such as a hospital backbone LAN and an Internet network.

The medical image diagnosis device 100 is, for example, an X-ray computed tomography (CT) device, and is typically installed in medical institutions, research facilities, and the like.

The learning device 200 receives information from each X-ray CT device (medical image diagnosis device) 100 via the communication network NW, and uses the received information to train a machine learning model MDL for removing noise from a medical image. Also, the learning device 200 transmits the trained machine learning model MDL to each X-ray CT device (medical image diagnosis device) 100 via the communication network NW.

The learning device 200 may be a single device or may be a system in which a plurality of devices connected via a communication network NW operate in cooperation with each other. That is, the learning device 200 may be implemented by a plurality of computers (processors) included in a distributed computing system or a cloud computing system. Moreover, the learning device 200 does not necessarily have to be a separate device different from the X-ray CT device (the medical image diagnosis device) 100 and may be a device integrated with the X-ray CT device (the medical image diagnosis device) 100.

### [Configuration of X-Ray CT Device]

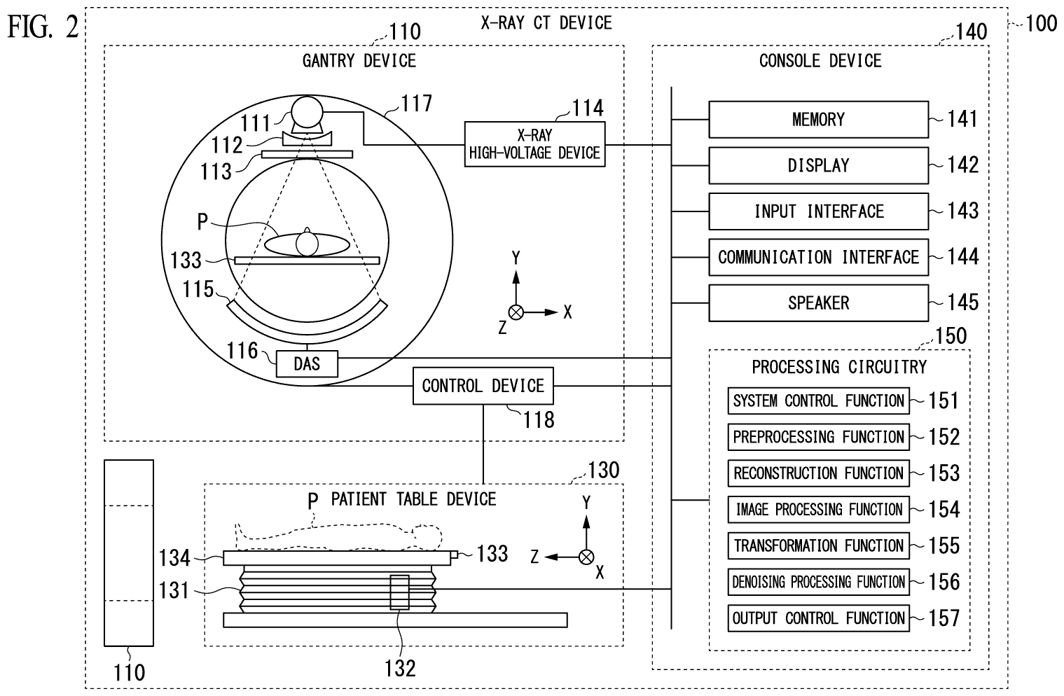

FIG. 2 is a diagram showing an example of a configuration of an X-ray CT device 100 in the first embodiment. The X-ray CT device 100 is a device that generates a medical image (hereinafter referred to as a CT image) of a subject P by scanning the subject P with X-rays, and diagnoses the subject P on the basis of the CT image. The subject P is typically a human being, but is not limited thereto, and may be other animals such as a dog or a cat, or may be a plant. Hereinafter, the subject P will be described as a human being as an example.

The X-ray CT device 100 may be, for example, a CT device using dual-energy CT (DECT) or photon-counting CT (PCCT). In the first embodiment, the X-ray CT device 100 will be described as a CT device using the DE.

As shown in FIG. 2, the X-ray CT device 100 includes, for example, a gantry device 110, a patient table device 130, and a console device 140. In the example shown in FIG. 2, for convenience of description, both a view of the gantry device 110 seen in a Z-axis direction and a view of the gantry device 110 seen in an X-axis direction are shown. However, in reality, there is only one gantry device 110. In the present embodiment, the rotation axis of the rotating frame 117 in a non-tilted state or the longitudinal direction of the tabletop 133 of the patient table device 130 is defined as a Z-axis direction, an axis perpendicular to the Z-axis direction and horizontal to a floor surface is defined as an X-axis direction, and a direction perpendicular to the Z-axis direction and vertical to the floor surface is defined as a Y-axis direction.

The gantry device 110 includes, for example, an X-ray tube 111, a wedge 112, a collimator 113, an X-ray high-voltage device 114, an X-ray detector 115, a data acquisition system (hereinafter, DAS) 116, a rotating frame 117, and a control device 118.

The X-ray tube 111 generates X-rays by radiating thermal electrons from a cathode (filament) to an anode (target) when a high voltage is applied from the X-ray high-voltage device 114. The X-ray tube 111 includes a vacuum tube. For example, the X-ray tube 111 is a rotating anode type X-ray tube that generates X-rays by irradiating a rotating anode with the thermal electrons.

The wedge 112 is a filter for adjusting the number of X-rays radiated from the X-ray tube 111 to the subject P. The wedge 112 attenuates the X-rays passing through the wedge 112 so that the distribution of the number of X-rays radiated from the X-ray tube 111 to the subject P becomes a predetermined distribution. The wedge 112 is also referred to as a wedge filter or a bow-tie filter. The wedge 112, for example, is made by processing aluminum so that a predetermined target angle and a predetermined thickness are formed.

The collimator 113 is a mechanism for narrowing down an irradiation range of the X-rays that have passed through the wedge 112. The collimator 113, for example, narrows down the irradiation range of the X-rays by forming slits by combining a plurality of lead plates. The collimator 113 may be referred to as an X-ray aperture. The X-ray high-voltage device 114 has, for example, a high-voltage generation device and an X-ray control device. The high voltage generation device has electric circuitry including a transformer, a rectifier, and the like and generates a high voltage to be applied to the X-ray tube 111. The X-ray control device controls an output voltage of the high-voltage generation device in accordance with the number of X-rays to be generated by the X-ray tube 111. The high-voltage generation device may boost the voltage with the above-described transformer or may boost the voltage with an inverter. The X-ray high-voltage device 114 may be provided on the rotating frame 117 or may be provided on a fixed frame (not shown) of the gantry device 110.

The X-ray detector 115 detects an intensity of incident X-rays generated by the X-ray tube 111 and passing through the subject P. The X-ray detector 115 outputs an electrical signal (which may be an optical signal or the like) according to the detected intensity of the X-rays to the DAS 116. The X-ray detector 115 has, for example, a plurality of X-ray detection element arrays. Each of the plurality of X-ray detection element arrays has a plurality of X-ray detection elements arrayed in a channel direction along an arc centered on a focal point of the X-ray tube 111. The plurality of X-ray detection element arrays are arrayed in a slice direction (a column direction or a row direction).

The X-ray detector 115 is an indirect detector having, for example, a grid, a scintillator array, and a photosensor array. The scintillator array has a plurality of scintillators. Each scintillator has a scintillator crystal. The scintillator crystal emits light of an amount of light according to the intensity of the incident X-rays. The grid is arranged on the surface of the scintillator array on which the X-rays are incident and has an X-ray shielding plate having a function of absorbing scattered X-rays. The grid may also be referred to as a collimator (a one-dimensional collimator or a two-dimensional collimator). The photosensor array has, for example, a photosensor such as a photomultiplier tube (PMT). The photosensor array outputs an electrical signal according to the amount of light emitted by the scintillator. The X-ray detector 115 may be a direct conversion type detector having a semiconductor element that converts the incident X-rays into an electrical signal.

The DAS 116 includes, for example, an amplifier, an integrator, and an analog-to-digital (A/D) converter. The amplifier performs an amplification process on the electrical signal output by each X-ray detection element of the X-ray detector 115. The integrator integrates the amplified electrical signal over a view period (to be described below). The A/D converter converts an electrical signal indicating an integration result into a digital signal. The DAS 116 outputs detection data based on the digital signal to the console device 140. The detection data is a digital value of an X-ray intensity identified by the channel number and row number of an X-ray detection element of a generation source, and the view number indicating the acquired view. The view number is a number that changes in accordance with the rotation of the rotating frame 117 and is, for example, a number that is incremented in accordance with the rotation of the rotating frame 117. Therefore, the view number is information indicating the rotation angle of the X-ray tube 111. The view period is a period that falls within the period from a rotation angle corresponding to a certain view number to a rotation angle corresponding to the next view number. The DAS 116 may detect the view switching according to a timing signal input from the control device 118, detect the view switching with an internal timer, or detect the view switching according to a signal acquired from a sensor (not shown). When a full scan is performed and X-rays are continuously radiated by the X-ray tube 111, the DAS 116 acquires a group of detection data for the entire circumference (360 degrees). When a half scan is performed and X-rays are continuously radiated by the X-ray tube 111, the DAS 116 acquires detection data for half the circumference (180 degrees).

The rotating frame 117 is an annular rotating member that rotates the X-ray tube 111, the wedge 112, the collimator 113, and the X-ray detector 115 in a state in which the X-ray tube 111, the wedge 112, and the collimator 113 are held facing the X-ray detector 115. The rotating frame 117 is supported by a fixed frame so that the rotating frame 117 is freely rotatable around the subject P introduced inside. The rotating frame 117 also supports the DAS 116. Detection data output by the DAS 116 is transmitted according to optical communication from a transmitter having a light-emitting diode (LED) provided on the rotating frame 117 to a receiver having a photodiode provided on a non-rotating part (e.g., the fixed frame) of the gantry device 110 using optical communication and is transferred to the console device 140 by the receiver. In addition, the method for transmitting detection data from the rotating frame 117 to the non-rotating part is not limited to the above-described method using optical communication, and any non-contact type transmission method may be adopted. The rotating frame 117 is not limited to an annular member, and may be an arm-like member as long as it can support and rotate the X-ray tube 111 or the like.

The control device 118 includes, for example, processing circuitry having a processor such as a central processing unit (CPU) and a drive mechanism including a motor, an actuator, and the like. The control device 118 receives an input signal from an input interface 143 attached to the console device 140 or the gantry device 110 and controls operations of the gantry device 110 and the patient table device 130.

The control device 118, for example, rotates the rotating frame 117, tilts the gantry device 110, and moves the tabletop 133 of the patient table device 130. When the gantry device 110 is tilted, the control device 118 rotates the rotating frame 117 around an axis parallel to the Z-axis direction on the basis of a tilt angle input to the input interface 143. The control device 118 acquires the rotation angle of the rotating frame 117 according to the output of a sensor (not shown) or the like. The control device 118 also outputs the rotation angle of the rotating frame 117 to the processing circuitry 150 at any time. The control device 118 may be provided in the gantry device 110 or the console device 140.

The control device 118 causes the gantry device 110 to perform a main scan photographing process or to perform a scanner photographing process which is a positioning/photographing process to be performed before the execution of the main scan photographing.

The patient table device 130 is a device on which the subject P to be scanned is placed and introduced into the rotating frame 117 of the gantry device 110. The patient table device 130 has, for example, a base 131, a patient table driving device 132, a tabletop 133, and a support frame 134. The base 131 includes a housing that supports the support frame 134 so that it is movable in a vertical direction (a Y-axis direction). The patient table driving device 132 includes a motor and an actuator. The patient table driving device 132 moves the tabletop 133, on which the subject P is placed, in a longitudinal direction of the tabletop 133 (a Z-axis direction) along the support frame 134. The tabletop 133 is a plate-shaped member on which the subject P is placed.

The console device 140 includes, for example, a memory 141 (storage circuitry), a display 142, an input interface 143, a communication interface 144, a speaker 145, and processing circuitry 150. Although a case where the console device 140 is separated from the gantry device 110 is described in the present embodiment, the gantry device 110 may include some or all of the constituent elements of the console device 140.

The memory 141 is implemented by, for example, a semiconductor memory element such as a random-access memory (RAM), a flash memory, a hard disk, an optical disk, or the like. Moreover, the memory 141 may include a storage medium such as a read-only memory (ROM) or a register.

The memory 141 stores, for example, detection data, projection data, reconstructed images, and the like. These data items may be stored in an external memory (for example, a network attached storage (NAS)) with which the X-ray CT device 100 can communicate, instead of the memory 141 (or in addition to the memory 141).

The memory 141 further stores model definition data. The model definition data is data such as a program or an algorithm that defines several trained models MDLα, MDLβ, MDLκ, and MDLλ, which will be described below. The trained models MDLα and MDLβ are examples of a “first trained model” and the trained models MDLκ and MDLλ are examples of a “second trained model.”

The display 142 displays various types of information. For example, the display 142 displays a CT image generated by the processing circuitry 150, a graphical user interface (GUI) that receives various types of operations from an operator, and the like. The operator is, for example, a medical professional such as a doctor, an engineer, or a nurse. The display 142 is, for example, a liquid crystal display, a CRT, an organic electroluminescence (EL) display, or the like.

The input interface 143 receives various types of input operations from the operator and outputs an electrical signal indicating the content of the received input operation to the processing circuitry 150.

For example, the input interface 143 is implemented by a pointing device (such as a mouse, a touch panel, a trackball, a joystick, a pen tablet, or a stylus), a keyboard, a switch, a button, a foot pedal, a camera, an infrared sensor, a microphone, or the like. In the present specification, the input interface 143 is not limited to an interface having physical operation parts such as a mouse and a keyboard. For example, an example of the input interface 143 also includes electrical signal processing circuitry that receives an electrical signal corresponding to an input operation from an external input device provided separately from the device and outputs the electrical signal to control circuitry.

The communication interface 144 includes, for example, a network interface card (NIC), a wireless communication module, or the like. The communication interface 144 communicates with an external device such as the learning device 200 via the communication network NW.

The speaker 145 outputs a sound on the basis of information output by the processing circuitry 150.

The processing circuitry 150 controls the overall operation of the X-ray CT device 100. The processing circuitry 150 executes, for example, a system control function 151, a preprocessing function 152, a reconstruction function 153, an image processing function 154, a transformation function 155, a denoising processing function 156, and an output control function 157. The processing circuitry 150 implements these functions with, for example, a hardware processor executing a program stored in the memory 141.

The DAS 116 and the preprocessing function 152 described above are examples of an “acquisition unit.” The reconstruction function 153 is an example of a “reconstruction unit.” The transformation function 155 is an example of a “transformation unit.” The denoising processing function 156 is an example of a “denoising processing unit.”

A hardware processor refers to circuitry such as a central processing unit (CPU), a graphics processing unit (GPU), an application-specific integrated circuit (ASIC), or a programmable logic device (e.g., a simple programmable logic device (SPLD), a complex programmable logic device (CPLD), or a field programmable gate array (FPGA)).

The program may be directly embedded in the circuitry of the hardware processor instead of storing the program in the memory 141. In this case, the hardware processor implements the function by reading and executing the program embedded in the circuitry.

The above-described program may be stored in advance in the memory 141. Alternatively, the above-described program may be stored in a non-transitory storage medium such as a DVD or CD-ROM and installed in the memory 141 from the non-transitory storage medium when the non-transitory storage medium is mounted on a drive device (not shown) of the console device 140.

The hardware processor is not limited to a configuration as single circuitry, but may be configured as a single hardware processor by combining a plurality of independent types of circuitry to implement each function. Moreover, a plurality of constituent elements may be integrated into a single hardware processor to implement each function.

The respective constituent elements of the console device 140 or the processing circuitry 150 may be distributed and implemented by a plurality of pieces of hardware. The processing circuitry 150 may not be a constituent element of the console device 140, but may be implemented by an external device (e.g., the learning device 200) capable of communicating with the console device 140. The external device, for example, may be a workstation connected to one X-ray CT device 100 or may be a device (e.g., a cloud server) connected to a plurality of X-ray CT devices 100 and configured to collectively execute processes equivalent to those of the processing circuitry 150.

The system control function 151 is executed to control various types of functions of the processing circuitry 150 on the basis of operations input to the input interface 143. Moreover, the system control function 151 is executed to acquire information from an external device such as the learning device 200 via the communication interface 144.

The preprocessing function 152 is executed to perform preprocessing on the detection data output by the DAS 116 to generate projection data and cause the memory 141 to store the generated projection data. The preprocessing includes various types of processing such as a logarithmic conversion process, an offset correction process, an inter-channel sensitivity correction process, and beam hardening correction.

The reconstruction function 153 is executed to perform a reconstruction process using a filtered back projection method, an iterative approximation reconstruction method, or the like on the projection data generated by the preprocessing function 152 to generate a CT image (also referred to as a reconstructed image) and cause the memory 141 to store the generated CT image (reconstructed image).

The image processing function 154 is executed to transform the CT image (including a denoised CT image to be described below) into a three-dimensional image or cross-sectional image data of any cross-section by a known method on the basis of an operation input to the input interface 143. The transformation into a three-dimensional image may be performed by the preprocessing function 152.

The transformation function 155 is executed to separate the reconstructed CT image into a signal image and a noise image by a whitening transform. The whitening transform is a type of linear transform and is a method for eliminating a correlation between a signal component and a noise component by using covariance analysis, eigenvalue decomposition, principal component analysis, independent principal component analysis, Luma-Chroma, or the like. The signal image is an image in which the signal components are significantly larger than the noise components among the signal components and the noise components contained in the CT image. The noise image is an image in which the noise components are significantly larger than the signal components among the signal components and the noise components contained in the CT image.

Furthermore, the transformation function 155 is executed to generate a denoised CT image by coupling the denoised signal image and the noise image, which will be described below, by an inverse whitening transform.

The denoising processing function 156 is executed to denoise (removes noise from) at least one of the signal image and the noise image separated from the CT image according to the transformation function 155. For example, the denoising processing function 156 may denoise noise from each image using a machine learning model MDL trained in advance (hereinafter also referred to as a trained model MDL). MDL is simply a symbol indicating a model. Details of the denoising process using the trained model MDL will be described below.

The output control function 157 is executed to control the display 142, the input interface 143, the communication interface 144, and the speaker 145 to output various types of information.

For example, the output control function 157 may cause the display 142 to display a denoised CT image or cause the display 142 to display a pre-denoising CT image. Furthermore, the output control function 157 may cause the display 142 to display a graphical user interface (GUI) for receiving various types of operations from an operator such as a doctor or an engineer.

Moreover, for example, the output control function 157 may be executed to transmit the denoised CT image or the pre-denoising CT image to an external device via the communication interface 144.

### [Processing Flow and Inference (Runtime) of X-Ray CT Device]

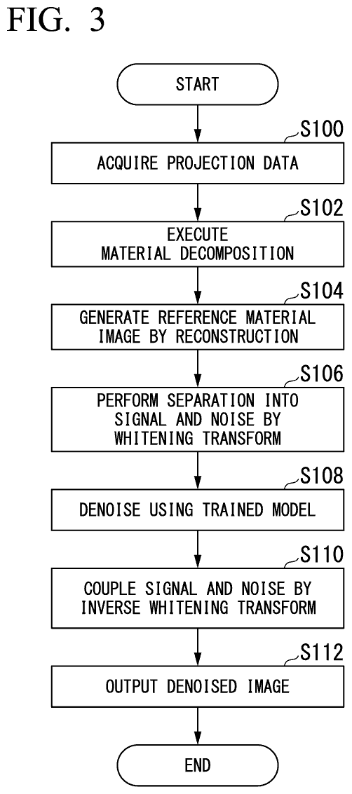

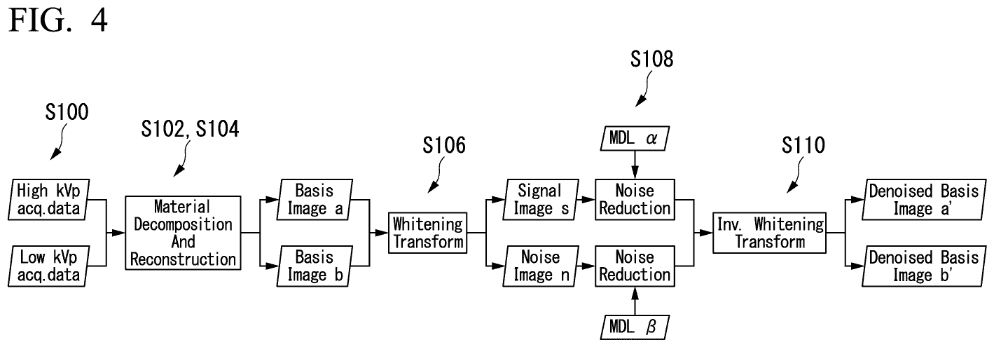

Hereinafter, a flow of a series of processing steps of the X-ray CT device 100 will be described with reference to FIGS. 3 and 4. FIG. 3 is a flowchart showing an example of the flow of the series of processing steps of the X-ray CT device 100 in the first embodiment. FIG. 4 is a diagram schematically showing a flow of a series of processing steps of the X-ray CT device 100 in the first embodiment. The process of the present flowchart is executed after the machine learning model MDL is trained.

First, the preprocessing function 152 is executed to perform preprocessing on the detection data output by the DAS 116 to acquire projection data (step S100).

The X-ray CT device 100 according to the first embodiment is a DECT device and photographs the subject P with two types of X-rays having different tube voltages, which are the voltages of the X-ray tube 111. Therefore, the projection data includes high-tube-voltage (kVp greater than or equal to a threshold value) projection data and low-tube-voltage (kVp less than the threshold value) projection data. The threshold value is, for example, about 120 kVp.

Subsequently, the reconstruction function 153 is executed to perform material decomposition on the basis of the acquired high-tube-voltage (high kVp) projection data and the acquired low-tube-voltage (low kVp) projection data (step S102).

For example, the reconstruction function 153 is executed to discriminate between the reference materials a and b from two types of projection data using attenuation coefficients of two different types of reference materials (also referred to as base materials) a and b, such as iodine and water. In addition, the number of reference materials is not limited to two and may be one or three or more.

Subsequently, the reconstruction function 153 is executed to perform a reconstruction process on the projection data subjected to material decomposition to generate a CT image (also referred to as a reference material image) in which each reference material is enhanced or suppressed (step S104). The reference material image is also referred to as a spectral image.

For example, the reconstruction function 153 is executed to perform a reconstruction process on the projection data in which the reference material a has been discriminated and generate a CT image in which the reference material a (e.g., iodine) has been emphasized. Likewise, the reconstruction function 153 is executed to perform a reconstruction process on the projection data in which the reference material b has been discriminated and generate a CT image in which the reference material b (e.g., water) has been emphasized. Hereinafter, the CT image in which the reference material a has been emphasized will be referred to as a “reference material image a” and the CT image in which the reference material b has been emphasized will be referred to as a “reference material image b.” The projection data in which the reference material a has been discriminated and the projection data in which the reference material b has been discriminated are examples of “reference material projection data.”

Subsequently, the transformation function 155 is executed to separate the reference material image into a signal image s and a noise image n by a whitening transform (step S106).

For example, when the reference material image a and the reference material image b are generated, the transformation function 155 is executed to separate the reference material image b into a signal image s and a noise image n while separating the reference material image a into a signal image s and a noise image n by the whitening transform.

Subsequently, the denoising processing function 156 is executed to denoise each of the signal image s and the noise image n using the trained model MDL (step S108).

As described above, the signal image s separated by the whitening transform is an image in which the signal components are significantly larger than the noise components, and the noise image n separated by the whitening transform is an image in which the noise components are significantly larger than the signal components. In other words, it is not possible to completely separate the signal and the noise by the whitening transform, the signal image s is mixed with noise components and the noise image n is mixed with signal components.

Therefore, the denoising processing function 156 uses the trained model MDL to remove the noise components from the signal image s and remove the signal components from the noise image n.

For example, the denoising processing function 156 is executed to read the first trained model MDLα and the second trained model MDLβ defined by the model definition data stored in the memory 141, input the signal image s to the first trained model MDLα, and input the noise image n to the second trained model MDLβ.

The first trained model MDLα and the second trained model MDLβ, for example, may be implemented by a deep neural network such as a convolutional neural network(s) (CNN). Instead of the neural network, the first trained model MDLα and the second trained model MDLβ may be implemented using other machine learning models such as a support vector machine, a decision tree, a random forest, and a logistic regression.

When the first trained model MDLα and the second trained model MDLβ are implemented by a neural network, the model definition data includes, for example, coupling information indicating how the units included in each of the layers constituting the neural network, such as the input layer, one or more hidden layers (intermediate layers), and the output layer, are coupled to each other, weight information indicating how many the coupling coefficients are assigned to data input and output between the coupled units, and the like.

The coupling information includes, for example, the number of units included in each layer, information for designating a type of unit to which each unit is coupled and information about an activation function that implements each unit, a gate provided between units in the hidden layer, and the like.

The activation functions that implement the units may be, for example, rectified linear unit (ReLU) functions, exponential linear units (ELU) functions, clipping functions, sigmoid functions, step functions, hyperbolic tangent functions, identity functions, and the like. The gates, for example, selectively transmit or weight data to be communicated between the units in accordance with a value (e.g., 1 or 0) returned by the activation function.

The coupling coefficients, for example, include weights that are assigned to output data when data is output from a unit in a certain layer to a unit in a deeper layer in a hidden layer of a neural network. Moreover, the coupling coefficients may include bias components specific to each layer.

The first trained model MDLα and the second trained model MDLβ are trained to output an image (i.e., a denoised image) in which noise has been reduced from a certain image when the certain image has been input.

Therefore, the denoising processing function 156 is executed to input the signal image s separated from the reference material image a to the first trained model MDLα and therefore acquire a denoised image of the signal image s separated from the reference material image a (hereinafter referred to as a denoised signal image s) from the first trained model MDLα.

Likewise, the denoising processing function 156 is executed to input the signal image s separated from the reference material image b to the first trained model MDLα and therefore acquire the denoised signal image s of the signal image s separated from the reference material image b from the first trained model MDLα.

Furthermore, the denoising processing function 156 is executed to input the noise image n separated from the reference material image a to the second trained model MDLβ and therefore acquire a denoised image of the noise image n separated from the reference material image a (hereinafter referred to as a denoised noise image n) from the second trained model MDLβ.

Likewise, the denoising processing function 156 is executed to input a noise image n separated from the reference material image b to the second trained model MDLβ and therefore acquire the denoised noise image n of the noise image n separated from the reference material image b from the second trained model MDLβ.

Subsequently, the transformation function 155 is executed to generate a denoised reference material image (hereinafter referred to as a denoised reference material image) by coupling the denoised signal image s and the denoised noise image n for each reference material image by an inverse whitening transform (step S110).

For example, the transformation function 155 is executed to generate a denoised reference material image a′ by coupling the denoised signal image s and the denoised noise image n derived from the reference material image a by an inverse whitening transform. Likewise, the transformation function 155 is executed to generate a denoised reference material image b′ by coupling the denoised signal image s and the denoised noise image n derived from the reference material image b by an inverse whitening transform.

Subsequently, the output control function 157 is executed to output the denoised image (step S112). For example, the output control function 157 may cause the display 142 to display the denoised reference material image a′ and the denoised reference material image b′. Moreover, the output control function 157 may be executed to transmit the denoised reference material image a′ and the denoised reference material image b′ to an external device (for example, a computer used by a doctor, an engineer, or the like) via the communication interface 144. Thereby, the process of the present flowchart ends.

### [Configuration of Learning Device]

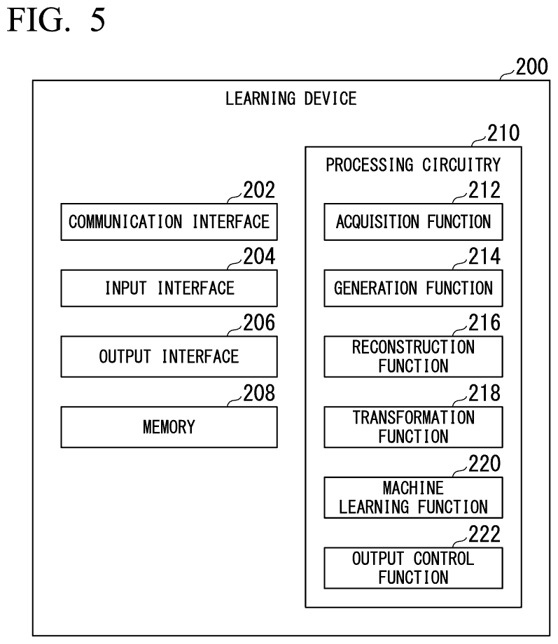

Hereinafter, a configuration of the learning device 200 that trains the machine learning model MDL will be described. FIG. 5 is a diagram showing an example of the configuration of the learning device 200 in the first embodiment. The learning device 200 includes, for example, a communication interface 202, an input interface 204, an output interface 206, a memory 208, and processing circuitry 210.

The communication interface 202 communicates with external devices via the communication network NW. The communication interface 202 includes, for example, an NIC and an antenna for wireless communication.

The input interface 204 receives various types of input operations from an operator, converts the received input operations into electrical signals, and outputs the electrical signals to the processing circuitry 210.

For example, the input interface 204 includes a mouse, a keyboard, a trackball, a switch, a button, a joystick, a touch panel, and the like. The input interface 204 may be, for example, a user interface that receives a sound input from a microphone or the like. When the input interface 204 is a touch panel, the input interface 204 may also have a display function of a display 213a included in the output interface 206 to be described below.

In addition, in the present specification, the input interface 204 is not limited to an interface having physical operation parts such as a mouse and a keyboard. For example, an example of the input interface 204 also includes electrical signal processing circuitry that receives an electrical signal corresponding to an input operation from an external input device provided separately from the device and outputs the electrical signal to control circuitry.

The output interface 206 includes, for example, the display 213a and a speaker 213b. The display 213a displays various types of information. For example, the display 213a displays an image generated by the processing circuitry 210, a GUI for receiving various types of input operations from the operator, and the like. For example, the display 213a is an LCD, a CRT display, an organic EL display, or the like. The speaker 213b outputs information input from the processing circuitry 210 as a sound.

The memory 208 is implemented by, for example, a semiconductor memory element such as a RAM or a flash memory, a hard disk, or an optical disk. These non-transitory storage media may be implemented by other storage devices connected via the communication network NW such as a NAS or an external storage server device.

Moreover, the memory 208 may include a non-transitory storage medium such as a ROM or a register. The memory 208 stores programs to be executed by the hardware processor of the processing circuitry 210, various types of calculation results of the processing circuitry 210, model definition data, and the like.

The processing circuitry 210 includes, for example, an acquisition function 212, a generation function 214, a reconstruction function 216, a transformation function 218, a machine learning function 220, and an output control function 222.

The processing circuitry 210 implements these functions by, for example, a hardware processor (a computer) executing a program stored in the memory 208 (storage circuitry).

The hardware processor in the processing circuitry 210 refers to circuitry such as a CPU, a GPU, an application-specific integrated circuit, a programmable logic device (e.g., a simple programmable logic device, a complex programmable logic device, or a field programmable gate array), or the like.

Instead of storing the program in memory 208, the program may be configured to be directly embedded within the circuitry of the hardware processor. In this case, the hardware processor implements the function by reading and executing the program embedded within the circuitry. The above-described program may be stored in the memory 208 in advance. Alternatively, the above-described program may be stored in a non-transitory storage medium such as a DVD or CD-ROM and installed in the memory 208 from the non-transitory storage medium when the non-transitory storage medium is mounted in a drive device (not shown) of the learning device 200.

The hardware processor is not limited to a configuration as single circuitry, but may be configured as a single hardware processor by combining a plurality of independent types of circuitry to implement each function. Moreover, a plurality of constituent elements may be integrated into a single hardware processor to implement each function.

### [Processing Flow Training of Learning Device]

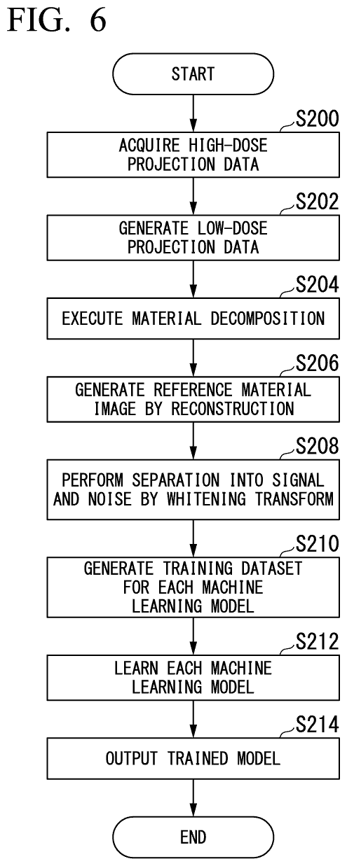

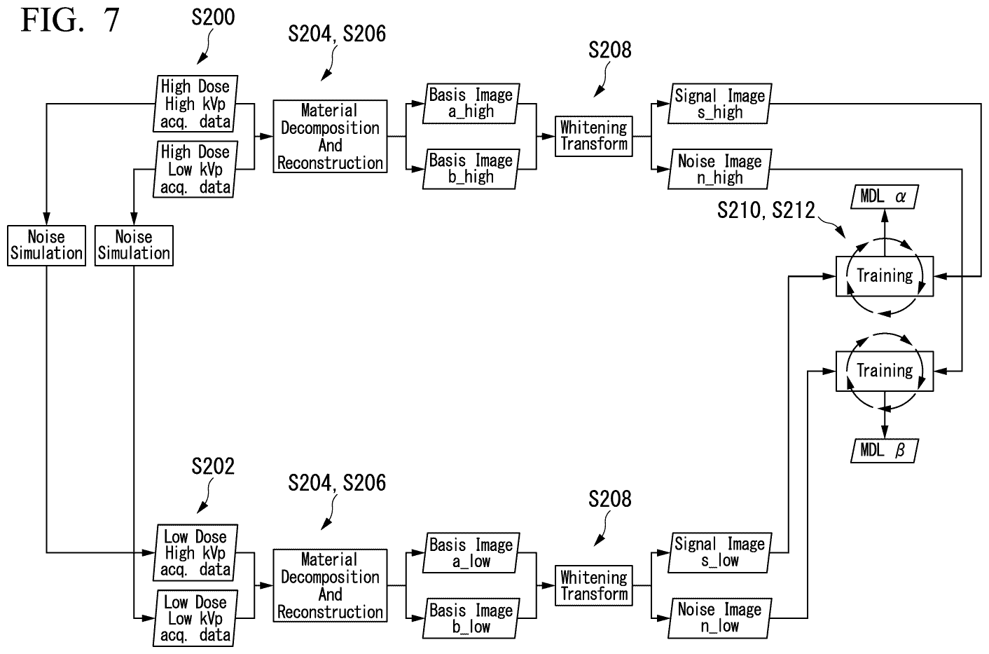

Hereinafter, a training process of the learning device 200 will be described with reference to FIGS. 6 and 7. FIG. 6 is a flowchart showing a flow of a series of processing steps of the learning device 200 according to the first embodiment. FIG. 7 is a diagram schematically showing a flow of a series of processing steps of the learning device 200 according to the first embodiment. The process of the present flowchart is executed when the machine learning model MDL is trained.

First, the acquisition function 212 is executed to acquire projection data in which the X-ray dose is greater than or equal to a threshold value (i.e., high-dose projection data) (step S200).

The acquisition function 212 is executed to acquire high-tube-voltage (high kVp) projection data and low-tube-voltage (low kVp) projection data as high-dose projection data.

For example, the acquisition function 212 may be executed to access the X-ray CT device 100 via the communication interface 202 and acquire high-dose projection data from the X-ray CT device 100. Moreover, when a user inputs the high-dose projection data to the input interface 204, the acquisition function 212 may be executed to acquire the high-dose projection data from the input interface 204. Furthermore, when the high-dose projection data is stored in the memory 208, the acquisition function 212 may be executed to acquire the high-dose projection data from the memory 208.

Subsequently, the generation function 214 is executed to generate projection data with an X-ray dose below a threshold value (i.e., low-dose projection data) from the high-dose projection data acquired by the acquisition function 212 (step S202).

For example, the generation function 214 may be executed to generate low-dose, high-tube-voltage (high kVp) projection data from high-dose, high-tube-voltage (high kVp) projection data according to noise simulation and generate low-dose, low-tube-voltage (low kVp) projection data from high-dose, low-tube-voltage (low kVp) projection data.

Subsequently, the reconstruction function 216 is executed to perform material decomposition on the basis of a combination of the high-dose, high-tube-voltage (high kVp) projection data and the high-dose, low-tube-voltage (low kVp) projection data and perform material decomposition on the basis of a combination of low-dose, high-tube-voltage (high kVp) projection data and low-dose, low-tube-voltage (low kVp) projection data (step S204).

For example, the reconstruction function 216 is executed to discriminate between the reference materials a and b from a combination of the high-dose, high-tube-voltage (high kVp) projection data and the high-dose, low-tube-voltage (low kVp) projection data and discriminate between the reference materials a and b from the combination of the low-dose, high-tube-voltage (high kVp) projection data and the low-dose, low-tube-voltage (low kVp) projection data using the attenuation coefficients of two different types of reference materials a and b.

Subsequently, the reconstruction function 216 is executed to perform a reconstruction process on the projection data subjected to the material decomposition and generate a reference material image, which is a CT image in which each reference material is enhanced or suppressed (step S206).

For example, the reconstruction function 216 is executed to perform a reconstruction process on high-dose projection data in which the reference materials a and b have been discriminated and generate a reference material image a_high and a reference material image b_high. Likewise, the reconstruction function 216 is executed to perform a reconstruction process on low-dose projection data in which the reference materials a and b have been discriminated and generate a reference material image a_low and a reference material image b_low.

Subsequently, the transformation function 218 is executed to separate each of the four types of reference material images into a signal image s and a noise image n by the whitening transform (step S208).

For example, the transformation function 218 is executed to separate the reference material image b_high into a signal image s_high and a noise image n_high while separating the reference material image a_high into a signal image s_high and a noise image n_high by the whitening transform. Likewise, the transformation function 155 is executed to separate the reference material image b_low into a signal image s_low and a noise image n_low while separating the reference material image a_low into a signal image s_low and a noise image n_low by the whitening transform.

Subsequently, the machine learning function 220 is executed to generate a training dataset for each trained model MDL (step S210).

For example, the machine learning function 220 is executed to generate a dataset in which a signal image s_high separated from a high-dosed-derived reference material image a_high is associated as a target with a signal image s_low separated from a low-dosed-derived reference material image a_low as a first training dataset or generate a dataset in which a signal image s_high separated from a high-dosed-derived reference material image b_high is associated as a target with a signal image s_low separated from a low-dose-derived reference material image b_low.

Moreover, the machine learning function 220 is executed to generate a dataset in which a noise image n_high separated from the high-dosed-derived reference material image a_high is associated as a target with a noise image n_low separated from the low-dosed-derived reference material image a_low as a second training dataset or generate a dataset in which the noise image n_high separated from the high-dosed-derived reference material image b_high is associated as a target with a noise image n_low separated from the low-dosed-derived reference material image b_low.

Subsequently, the machine learning function 220 is executed to train each trained model MDL using the training dataset (step S212).

For example, the machine learning function 220 is executed to train the first trained model MDLα using the first training dataset. Specifically, the machine learning function 220 is executed to input the signal image s_low to the first trained model MDLα. The machine learning function 220 is executed to calculate a difference between the image output by the first trained model MDLα in accordance with the input of the signal image s_low and the target signal image s_high. Also, the machine learning function 220 is executed to adjust parameters (a weight coefficient and a bias component) of the first trained model MDLα by performing error backpropagation using a stochastic gradient descent method or the like on the basis of the difference.

Furthermore, the machine learning function 220 is executed to train the second trained model MDLβ using the second training dataset. Specifically, the machine learning function 220 is executed to input the noise image n_low to the second trained model MDLβ. The machine learning function 220 is executed to calculate a difference between the image output by the second trained model MDLβ in accordance with the input of the noise image n_low and the target noise image n_high. Also, the machine learning function 220 is executed to adjust parameters (a weight coefficient and a bias component) of the second trained model MDLβ by performing error backpropagation using a stochastic gradient descent method or the like on the basis of the difference.

Thus, the machine learning function 220 is executed to iterate the above-described process until the number of training iterations reaches a specified number and store model definition data in which the first trained model MDLα and the second trained model MDLβ are defined in the memory 208 when the number of iterations reaches the specified number.

Subsequently, the output control function 222 is executed to transmit model definition data in which the first trained model MDLα and the second trained model MDLβ trained by the machine learning function 220 are defined to each X-ray CT device 100 via the communication interface 202 (step S214). Thereby, the process of the present flowchart ends.

According to the above-described first embodiment, the X-ray CT device 100 acquires high-tube-voltage (high kVp) projection data and low-tube-voltage (low kVp) projection data of X-rays radiated to the subject P, discriminates between the reference materials a and b on the basis of the high-tube-voltage (high kVp) projection data and the low-tube-voltage (low kVp) projection data, and generates reference material images a and b by the reconstruction process.

The X-ray CT device 100 separates each of the reference material images a and b into a signal image s and a noise image n by the whitening transform. The X-ray CT device 100 inputs the signal image s to the first trained model MDLα and inputs the noise image n to the second trained model MDLβ. The X-ray CT device 100 acquires a denoised signal image s from the first trained model MDLα and acquires a denoised noise image n from the second trained model MDLβ.

The first trained model MDLα is a machine learning model trained on the basis of a first training dataset including a dataset in which a signal image s_high separated from a high-dose-derived reference material image a_high is associated as a target with a signal image s_low separated from a low-dose-derived reference material image a_low or a dataset in which a signal image s_high separated from a high-dose-derived reference material image b_high is associated as a target with a signal image s_low separated from a low-dose-derived reference material image b_low.

The second trained model MDLβ is a machine learning model trained on the basis of a second training dataset including a dataset in which a noise image n_high separated from the high-dose-derived reference material image a_high is associated as a target with a noise image n_low separated from the low-dose-derived reference material image a low or a dataset in which a noise image n_high separated from the high-dose-derived reference material image b_high is associated as a target with a noise image n_low separated from the low-dose-derived reference material image b_low.

The X-ray CT device 100 generates denoised reference material images a′ and b′ by coupling the denoised signal image s and the denoised noise image n for each reference material image by an inverse whitening transform. Also, the X-ray CT device 100, for example, causes the display 142 to display the denoised reference material images a′ and b′ or transmits the denoised reference material images a′ and b′ to an external device via the communication interface 144.

In other words, the X-ray CT device 100 (i) separates the reference material image into a signal image s and a noise image n by the whitening transform, (ii) denoises each of the signal image s and the noise image n using the trained model MDL, and (iii) couples the denoised signal image s and the denoised noise image n by the inverse whitening transform, thereby generating a denoised reference material image.

As described above, by the whitening transform, the signal cannot be completely separated from the noise. The signal image s is mixed with noise components and the noise image n is mixed with signal components. Therefore, the trained model MDL is used to remove the noise components from the signal image s and remove the signal components from the noise image n, and then couple them to generate a denoised reference material image. By performing this process, the accuracy of noise reduction in spectral imaging can be improved.

### Modified Example of First Embodiment

Hereinafter, a modified example of the first embodiment will be described. Although the medical image generated in the reconstruction process is referred to as a reference material image in the above-described first embodiment, the present invention is not limited thereto. For example, a virtual monochromatic X-ray image, a virtual plain image, an iodine map, an effective atomic number image, or an electron density image may be generated in the reconstruction process. In this case, it is assumed that the medical images included in each training dataset are also these virtual monochromatic X-ray images, virtual plain images, iodine maps, effective atomic number images, or electron density images.

### Second Embodiment

Hereinafter, a second embodiment will be described. In the above-described first embodiment, the X-ray CT device 100 has been described as a CT device using dual-energy CT (DECT). On the other hand, the second embodiment is different from the first embodiment in that the X-ray CT device 100 is a photon-counting CT (PCCT) device. Hereinafter, the differences from the first embodiment will be mainly described and the description of portions identical to those in the first embodiment will be omitted. In addition, in the description of the second embodiment, parts identical to those in the first embodiment are denoted by the same reference signs.

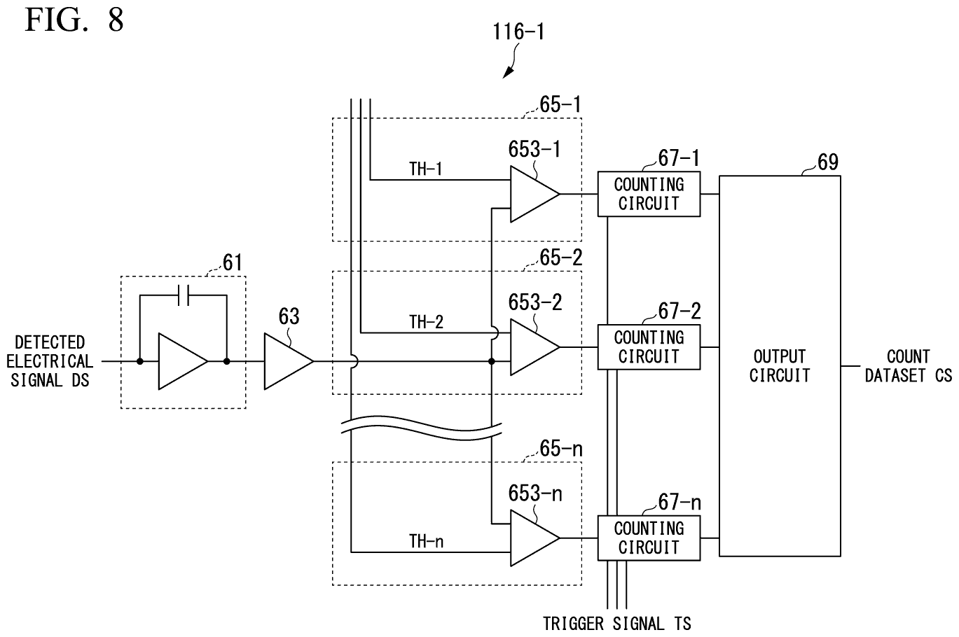

FIG. 8 is a diagram showing an example of a configuration of the DAS 116 of the X-ray CT device 100 according to the second embodiment. The DAS 116 of the X-ray CT device 100, which is a photon-counting CT device, has readout channels equal in number to channels corresponding to the number of X-ray detection elements. These multiple readout channels are implemented in parallel with an integrated circuit such as an application-specific integrated circuit (ASIC). FIG. 8 shows only a configuration of a DAS 116-1 for one readout channel.

The DAS 116-1 has a preamplifier circuit 61, a waveform shaping circuit 63, a plurality of pulse height discrimination circuits 65, a plurality of counting circuits 67, and an output circuit 69. The preamplifier circuit 61 amplifies a detected electrical signal DS (an electric current signal) from an X-ray detection element that is a connection destination. For example, the preamplifier circuit 61 converts the electric current signal from the X-ray detection element that is the connection destination into a voltage signal having a voltage value (a peak value) proportional to an amount of charge of the electric current signal. The waveform shaping circuit 63 is connected to the preamplifier circuit 61. The waveform shaping circuit 63 shapes the waveform of the voltage signal from the preamplifier circuit 61. For example, the waveform shaping circuit 63 reduces a pulse width of the voltage signal from the preamplifier circuit 61.

A number of counting channels corresponding to the number of energy bands (energy bins) are connected to the waveform shaping circuit 63. When n energy bins are set, n counting channels are provided in the waveform shaping circuit 63. Each counting channel has a pulse height discrimination circuit 65-n and a counting circuit 67-n.

Each of the pulse height discrimination circuits 65-n discriminates the energy of the X-ray photons detected by the X-ray detection element, which is a peak value of the voltage signal from the waveform shaping circuit 63. For example, the pulse height discrimination circuit 65-n has a comparison circuit 653-n. A voltage signal from the waveform shaping circuit 63 is input to one input terminal of each of the comparison circuits 653-n. A reference signal TH (a reference voltage value) corresponding to a different threshold value is supplied from the control device 118 to the other input terminal of each of the comparison circuits 653-n. For example, a comparison circuit 653-1 for an energy bin bin1 is supplied with a reference signal TH-1, a comparison circuit 653-2 for an energy bin bin2 is supplied with a reference signal TH-2, and a comparison circuit 653-n for an energy bin binn is supplied with a reference signal TH-n. Each of the reference signals TH has an upper limit reference value and a lower limit reference value. Each of the comparison circuits 653-n outputs an electric pulse signal when the voltage signal from the waveform shaping circuit 63 has a peak value corresponding to the energy bin corresponding to each of the reference signals TH. For example, the comparison circuit 653-1 outputs an electric pulse signal when the peak value of the voltage signal from the waveform shaping circuit 63 is a peak value corresponding to the energy bin bin1 (when it is between the reference signals TH-1 and TH-2). On the other hand, the comparison circuit 653-1 for the energy bin bin1 does not output an electric pulse signal when the peak value of the voltage signal from the waveform shaping circuit 63 is not a peak value corresponding to the energy bin bin1. Moreover, for example, the comparison circuit 653-2 outputs an electric pulse signal when the peak value of the voltage signal from the waveform shaping circuit 63 is a peak value corresponding to the energy bin bin2 (when it is between the reference signals TH-2 and TH-3).

The counting circuit 67-n counts the electric pulse signals from the pulse height discrimination circuit 65-n at a readout period that coincides with the view switching period. For example, the counting circuit 67-n is supplied with a trigger signal TS from the control device 118 at a switching timing of each view. By taking the opportunity that the trigger signal TS is supplied, the counting circuit 67-n adds 1 to the count number stored in the internal memory every time an electric pulse signal is input from the pulse height discrimination circuit 65-n. By taking the opportunity that the next trigger signal is supplied, the counting circuit 67-n reads data of a count number accumulated in the internal memory (i.e., count data) and supplies the read data to the output circuit 69. Moreover, the counting circuit 67-n resets the count number accumulated in the internal memory to an initial value every time the trigger signal TS is supplied. Thus, the counting circuit 67-n counts the count number for each view.

The output circuit 69 is connected to the counting circuits 67-n for a plurality of readout channels mounted on the X-ray detector 115. The output circuit 69 integrates count data from the counting circuits 67-n for a plurality of readout channels with respect to each of a plurality of energy bins to generate count data for a plurality of readout channels for each view. The count data for each energy bin is a collection of data with a count number defined by a channel, a segment (column), and an energy bin. The count data for each energy bin is transmitted to the console device 140 on a view-by-view basis. The count data for each view is referred to as a count dataset CS.

The DAS 116 having such a configuration acquires count data indicating a count number (a count value) of X-ray photons detected by the X-ray detector 115 for each set energy bin and acquires the count data for each energy bin as detection data.

The system control function 151 according to the second embodiment, for example, may be executed to set an energy bin to be referred to by the reconstruction function 153 on the basis of an input operation received by the input interface 143. The system control function 151 may be executed to automatically set an energy bin, regardless of an input operation received by the input interface 143.

The preprocessing function 152 according to the second embodiment is executed to generate projection data from the count data of each energy bin by performing predetermined preprocessing on the detection data (count data) output by the DAS 116. In other words, the preprocessing function 152 is executed to generate projection data for each energy bin. The predetermined preprocessing may include, for example, a logarithmic conversion process, an offset correction process, an inter-channel sensitivity correction process, beam hardening correction, scattered radiation correction, dark count correction, and the like.

The reconstruction function 153 is executed to perform a predetermined reconstruction process on the projection data generated by the preprocessing function 152 and generate a CT image from the projection data.

For example, the reconstruction function 153 may be executed to generate a CT image for each energy bin (hereinafter referred to as a bin image) by reconstructing projection data for each energy bin.

Moreover, the reconstruction function 153 may be executed to accumulate the projection data for each energy bin, calculate an amount of X-ray absorption on the basis of a sum of the projection data and a response function stored in the memory 141, and generate a counting image (also referred to as an integral image) on the basis of the amount of X-ray absorption.

Moreover, as in the first embodiment, the reconstruction function 153 may be executed to extract only the components of the reference material (e.g., iodine) from the projection data of each energy bin and reconstruct the projection data of each energy bin from which the components of the reference material have been extracted, thereby generating a CT image (a reference material image) in which the reference material is emphasized.

Furthermore, in addition to the reference material image, the reconstruction function 153 may be executed to generate virtual monochromatic X-ray images, virtual plain images, iodine maps, effective atomic number images, electron density images, CT images reconstructed for each energy bin (bin images), and the like.

Moreover, when a normal resolution (NR) mode and a super high resolution (SHR) mode are set in the X-ray CT device 100, which is a PCCT, the reconstruction function 153 may be executed to generate a CT image with a resolution corresponding to each mode. The NR mode is a mode in which the resolution is less than or equal to a threshold value and the SHR mode is a mode in which the resolution exceeds the threshold value. A CT image generated in the NR mode is also referred to as an NR image and a CT image generated in the SHR mode is also referred to as an SHR image.

The predetermined reconstruction process may include, for example, a filtered back projection method, an iterative approximation reconstruction method, and the like. The reconstruction function 153 stores the reconstructed CT image in the memory 141. When the preprocessing function 152 does not perform preprocessing, the reconstruction function 153 may be executed to perform the reconstruction process using the detection data (count data).

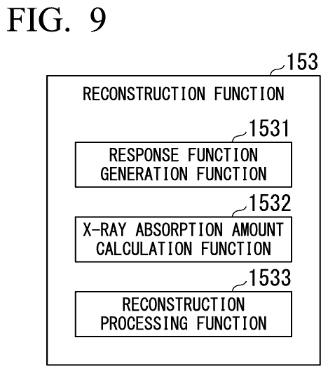

FIG. 9 is a diagram showing an example of a functional configuration of the reconstruction function 153 according to the second embodiment. The reconstruction function 153 includes, for example, a response function generation function 1531, an X-ray absorption amount calculation function 1532, and a reconstruction processing function 1533.

The response function generation function 1531 is executed to generate data of a response function indicating a detector response characteristic. For example, the response function generation function 1531 is executed to measure a response (i.e., detection energy and detection intensity) of a standard detection system for a plurality of monochromatic X-rays having a plurality of incident X-ray energies according to a combination of predictive calculation, experiment, and predictive calculation and experiment and generate a response function on the basis of measured values of the detection energy and detection intensity. Moreover, the response function generation function 1531 may be executed to generate data of the response function on the basis of actual measurement values acquired in calibration or the like. The response function specifies a relationship between the detection energy for each incident X-ray and the output response of the system. For example, the response function specifies a relationship between the detection energy for each incident X-ray and the detection intensity. The generated data of the response function is stored in the memory 141.

The X-ray absorption amount calculation function 1532 is executed to calculate the amount of X-ray absorption for each of the plurality of reference materials on the basis of count data for a plurality of energy bins included in the projection data, the energy spectrum of the X-rays incident on the subject P, and the response function stored in the memory 141. The X-ray absorption amount calculation function 1532 can be executed to calculate the amount of X-ray absorption without the influence of the response characteristics of the X-ray detector 115 and the DAS 116 by using the response function and calculating the amount of X-ray absorption on the basis of the count data and the energy spectrum of the X-rays incident on the subject P. As described in the first embodiment, a process for obtaining the amount of X-ray absorption for each reference material in this way is also referred to as material decomposition. The reference material can be set to any material such as calcium, calcification, bone, fat, muscle, air, organ, lesion, hard tissue, soft tissue, or contrast material. A type of reference material to be calculated may be decided in advance by the operator or the like via the input interface 143. The amount of X-ray absorption indicates the number of X-rays absorbed by the reference material. For example, the amount of X-ray absorption is specified by a combination of an X-ray attenuation coefficient and an X-ray transmission path length.

The reconstruction processing function 1533 is executed to reconstruct a photon-counting CT image expressing the spatial distribution of the reference material to be imaged among the plurality of reference materials on the basis of the amount of X-ray absorption related to each of the plurality of reference materials calculated by the X-ray absorption amount calculation function 1532, and cause the memory 41 to store the generated CT image. The reference material to be imaged may be of one or more types. The type of reference material to be imaged may be decided by the operator or the like via the input interface 143.

The projection data obtained by the photon-counting CT device includes information about the energy of X-rays attenuated by passing through the subject P. Therefore, the reconstruction processing function 1533, for example, can be executed to reconstruct a CT image of a specific energy component. Moreover, the reconstruction processing function 1533 can be executed to reconstruct a CT image of each of a plurality of energy components. Furthermore, the reconstruction processing function 1533, for example, can be executed to assign a color tone according to the energy component to each pixel of the CT image of each energy component, and superimpose a plurality of CT images color-coded according to the energy component.

The X-ray CT device 100, which is a photon-counting CT device, may generate a reference material image, a virtual monochromatic X-ray image, a virtual plain image, an iodine map, an effective atomic number image, and an electron density image, or may generate an NR image or an SHR image, in addition to or instead of generating a bin image or a counting image through a reconstruction process, as in the above-described first embodiment.

When a bin image is generated for each energy bin by the X-ray CT device 100, the learning device 200 according to the second embodiment provides machine learning models MDL equal in number to energy bins and trains each machine learning model MDL. For example, when there are four energy bins, the number of machine learning models MDL is four. Moreover, when a counting image is generated by the X-ray CT device 100, the learning device 200 provides and trains one machine learning model MDL regardless of the number of energy bins.

According to the above-described second embodiment, when the X-ray CT device 100 is a photon-counting CT device, the machine learning model MDL can be trained using a training dataset in which a reference material image, a virtual monochromatic X-ray image, a virtual plain image, an iodine map, an effective atomic number image, an electron density image, a bin image, a counting image, an NR image, and an SHR image are adopted.

### Third Embodiment

Hereinafter, a third embodiment will be described. As described in the above-described first and second embodiments, the signal image s between the signal image s and the noise image n separated from the reference material image by the whitening transform is denoised using the first trained model MDLα and the noise image n is denoised using the second trained model MDLβ.

In contrast, the third embodiment is different from the first and second embodiments in that both the signal image s and the noise image n are denoised using either the first trained model MDLα or the second trained model MDLβ. Hereinafter, the differences from the first and second embodiments will be mainly described and the description of portions identical to those in the first and second embodiments will be omitted. In addition, in the description of the third embodiment, parts identical to those in the first and second embodiments are denoted by the same reference signs.

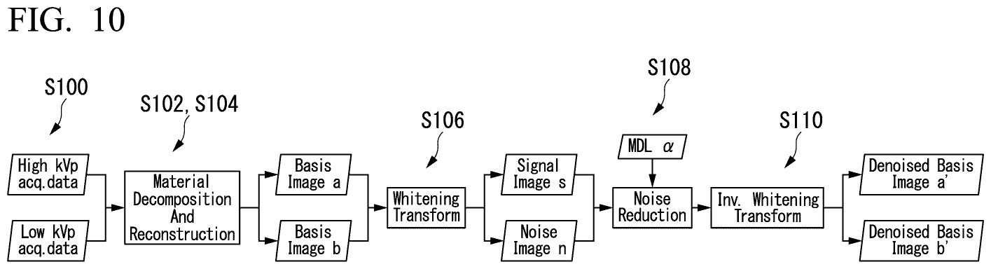

FIG. 10 is a diagram schematically showing a flow of a series of processing steps in the X-ray CT device 100 in the third embodiment.

The denoising processing function 156 according to the third embodiment, for example, is executed to denoise each of the signal image s and the noise image n by using either the first trained model MDLα or the second trained model MDLβ as the processing of step S108. Although a case where the first trained model MDLα is used as an example will be described in the third embodiment, the other second trained model MDLβ may be used.

Specifically, the denoising processing function 156 is executed to input the signal image s separated from the reference material image a to the first trained model MDLα and therefore acquire a denoised signal image s of the reference material image a from the first trained model MDLα.

Likewise, the denoising processing function 156 is executed to input the signal image s separated from the reference material image b to the first trained model MDLα and therefore acquire a denoised signal image s of the reference material image b from the first trained model MDLα.

Furthermore, the denoising processing function 156 is executed to input the noise image n separated from the reference material image a to the first trained model MDLα and therefore acquire a denoised noise image n of the reference material image a from the first trained model MDLα.

Likewise, the denoising processing function 156 is executed to input the noise image n separated from the reference material image b to the first trained model MDLα and therefore acquire a denoised noise image n of the reference material image b from the first trained model MDLα.

Thus, in the third embodiment, both the signal image s and the noise image n are denoised by inputting both the signal image s and the noise image n to the first trained model MDLα.

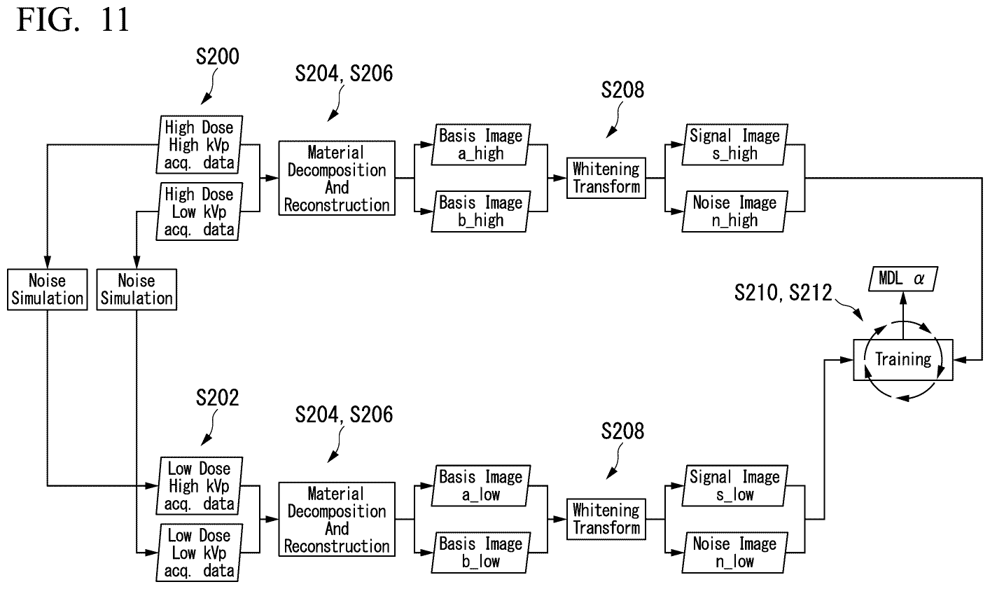

FIG. 11 is a diagram schematically showing a flow of a series of processing steps of the learning device 200 according to the third embodiment.

The machine learning function 220 according to the third embodiment is executed to generate a first training dataset for training the first trained model MDLα as the processing of step S210. For example, the machine learning function 220 is executed to generate datasets (i) to (iv) as the first training dataset.
(i) A dataset in which the signal image s_high separated from the high-dose-derived reference material a high is associated as a target with the signal image s_low separated from the low-dose-derived reference material image a_low.
(ii) A dataset in which the signal image s_high separated from the high-dose-derived reference material image b_high is associated as a target with the signal image s_low separated from the low-dose-derived reference material image b_low.
(iii) A dataset in which the noise image n_high separated from the high-dose-derived reference material image a high is associated as a target with the noise image n_low separated from the low-dose-derived reference material image a_low.
(iv) A dataset in which the noise image n_high separated from the high-dose-derived reference material image b_high is associated as a target with the noise image n_low separated from the low-dose-derived reference material image b_low.

As the processing of step S212, the machine learning function 220 is executed to train a first trained model MDLα using a first training dataset including (i) to (iv).

According to the above-described third embodiment, the X-ray CT device 100 separates each of the reference material image a and the reference material image b into the signal image s and the noise image n by the whitening transform. The X-ray CT device 100 inputs the signal image s and the noise image n to a first trained model MDLα trained on the basis of a first training dataset and acquires a denoised signal image s and a denoised noise image n from the first trained model MDLα.

In the above-described first or second embodiment, the first training dataset includes only the datasets (i) and (ii) derived from the signal image s. In contrast, in the third embodiment, the first training dataset further includes the datasets (iii) and (iv) derived from the noise image n, in addition to the datasets (i) and (ii) derived from the signal image s. By solely using the first trained model MDLα trained using this first training dataset, it is possible to denoise not only the signal image s but also the noise image n.

Although the first trained model MDLα is solely used to denoise both the signal image s and the noise image n in the above-described third embodiment, the present invention is not limited thereto. For example, the second trained model MDLβ may be solely used to denoise both the signal image s and the noise image n. In this case, a second training dataset including (i) to (iv) is used to train the second trained model MDLβ.

### Fourth Embodiment

Hereinafter, a fourth embodiment will be described. The fourth embodiment is different from the first to third embodiments in that either the signal image s or the noise image n is denoised using either the first trained model MDLα or the second trained model MDLβ. Hereinafter, the differences from the first to third embodiments will be mainly described and the description of portions identical to those in the first to third embodiments will be omitted. In addition, in the description of the fourth embodiment, parts identical to those in the first to third embodiments are denoted by the same reference signs.

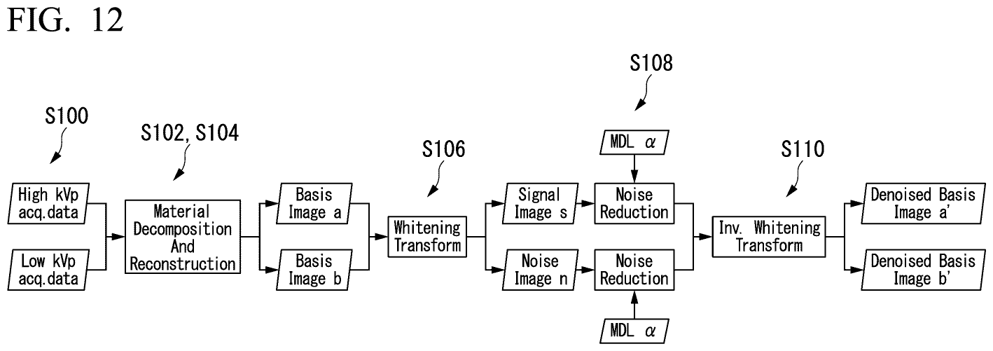

FIG. 12 is a diagram schematically showing a flow of a series of processing steps in the X-ray CT device 100 in the fourth embodiment.

The denoising processing function 156 according to the fourth embodiment, for example, is executed to denoise only one of the signal image s and the noise image n using either the first trained model MDLα or the second trained model MDLβ as the processing of step S108. In the fourth embodiment, as an example, a case where only the signal image s is denoised using only the first trained model MDLα will be described. In addition, only the noise image n may be denoised using only the first trained model MDLα. Moreover, only the signal image s or only the noise image n may be denoised using only the second trained model MDLβ.

For example, the denoising processing function 156 is executed to input the signal image s separated from the reference material image a to the first trained model MDLα and therefore acquire the denoised signal image s of the reference material image a from the first trained model MDLα.

Furthermore, the denoising processing function 156 is executed to input the noise image n separated from the reference material image a to the first trained model MDLα and therefore acquire a denoised noise image n of the reference material image a from the first trained model MDLα.

Subsequently, as the processing of step S110, the transformation function 155 according to the fourth embodiment is executed to generate a denoised reference material image by coupling the denoised signal image s and the noise image n for each reference material image by the inverse whitening transform.

For example, the transformation function 155 is executed to generate a denoised reference material image a′ by coupling the denoised signal image s and the noise image n derived from the reference material image a by the inverse whitening transform. Likewise, the transformation function 155 is executed to generate a denoised reference material image b′ by coupling the denoised signal image s and the noise image n derived from the reference material image b by the inverse whitening transform.

Thus, in the fourth embodiment, only the signal image s is denoised by inputting only the signal image s to the first trained model MDLα.

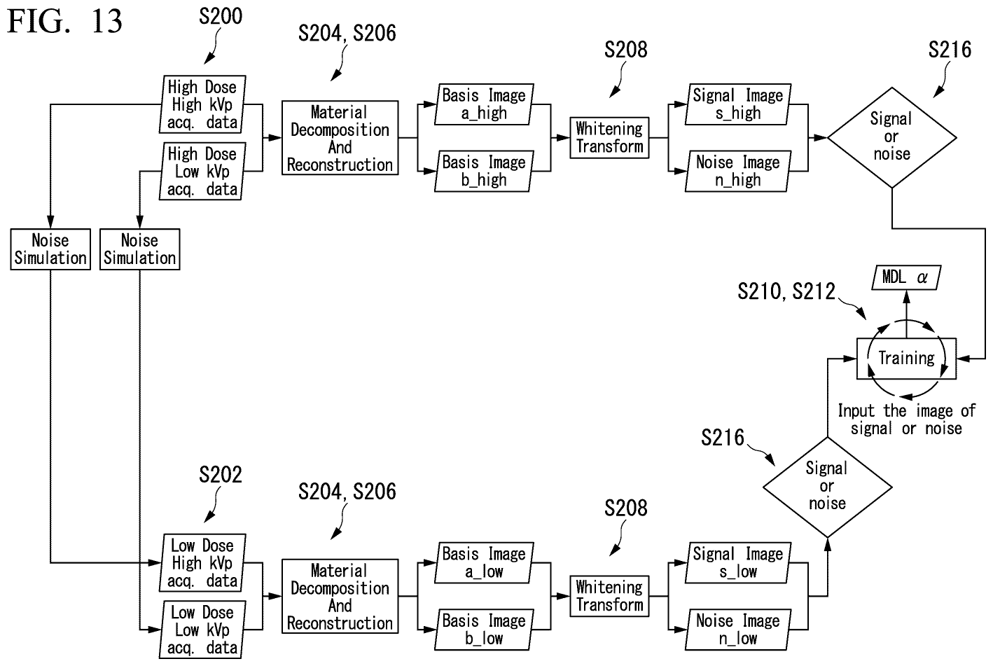

FIG. 13 is a diagram schematically showing a flow of a series of processing steps of the learning device 200 according to the fourth embodiment.

In the machine learning function 220 of the fourth embodiment, if the transformation function 218 is executed to separate each of the four types of reference material images into a signal image s and a noise image n by the whitening transform as the processing in S208, it is determined whether an image for which denoising is requested (hereinafter referred to as a denoising request image) is the signal image s or the noise image n (step S216).

For example, the denoising request image may be decided in accordance with the user's operation on the input interface 204. Moreover, the denoising request image may be received by the communication interface 202 from the X-ray CT device 100. Moreover, the denoising request image may be programmed in advance. In the fourth embodiment, as described above, it is assumed that the request image is a signal image s.

When the denoising request image is the signal image s, the machine learning function 220 is executed to extract only signal image s from a plurality of signal images s and a plurality of noise images n and generate a first training dataset including only the signal image s as the processing of step S210. For example, the machine learning function 220 is executed to generate the first training dataset including the above-described (i) and (ii).

As the processing of step S212, the machine learning function 220 is executed to train a first trained model MDLα using the first training dataset including (i) and (ii).

According to the above-described fourth embodiment, the X-ray CT device 100 separates the reference material image into a signal image s and a noise image n by the whitening transform. The X-ray CT device 100 inputs the signal image s to the first trained model MDLα trained on the basis of the first training dataset and acquires a denoised signal image s from the first trained model MDLα. Also, the X-ray CT device 100 couples the denoised signal image s and the noise image n by the inverse whitening transform to generate a denoised reference material image. Thus, the denoised reference material image can be generated by denoising only one of the signal image s and the noise image n without denoising both of them.

Although the first trained model MDLα is solely used to denoise the signal image s in the above-described fourth embodiment, the present invention is not limited thereto. For example, the second trained model MDLβ may be solely used to denoise the signal image s.

### Fifth Embodiment

Hereinafter, a fifth embodiment will be described. As described in the above-described first to fourth embodiments, the signal image s and/or the noise image n are denoised. In contrast, the fifth embodiment is different from the first to fourth embodiments in that not only the signal image s and/or the noise image n are denoised, but also the reference material image is denoised.

Hereinafter, the differences from the first to fourth embodiments will be mainly described and the description of portions identical to those in the first to fourth embodiments will be omitted. In addition, in the description of the fifth embodiment, parts identical to those in the first to fourth embodiments are denoted by the same reference signs.

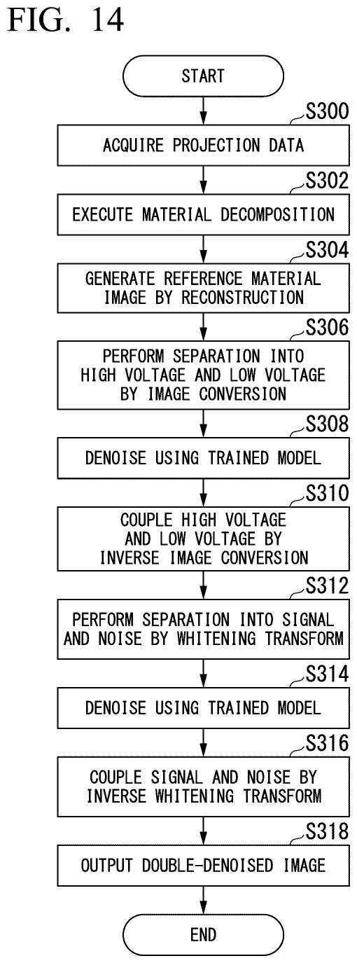

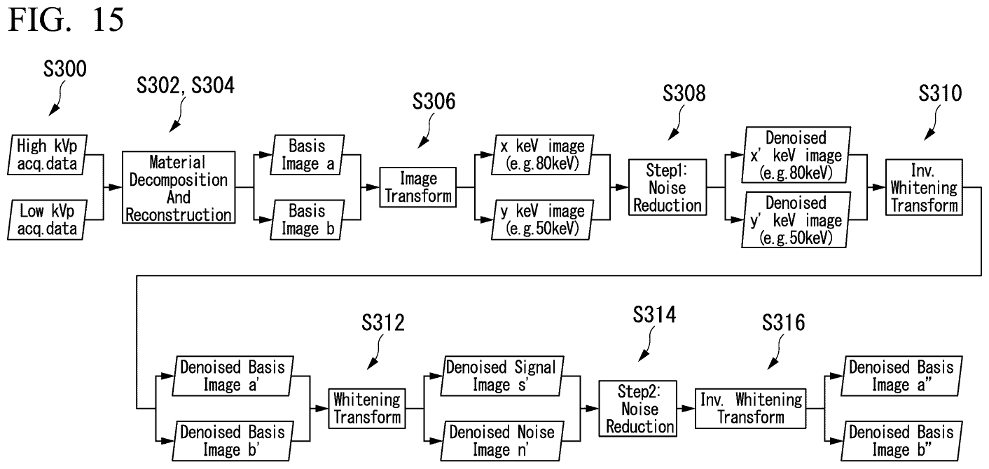

FIG. 14 is a flowchart showing an example of a flow of a series of processing steps of the X-ray CT device 100 in the fifth embodiment. FIG. 15 is a diagram schematically showing a flow of a series of processing steps of the X-ray CT device 100 in the fifth embodiment. The process of the present flowchart is executed after the machine learning model MDL is trained.

First, the preprocessing function 152 is executed to perform preprocessing on detection data output by the DAS 116 and acquire projection data (step S300). The acquired projection data includes high-tube-voltage (kVp greater than or equal to a threshold value) projection data and low-tube-voltage (kVp less than the threshold value) projection data.

Subsequently, the reconstruction function 153 is executed to perform material decomposition on the basis of the acquired high-tube-voltage (high kVp) projection data and the acquired low-tube-voltage (low kVp) projection data (step S302).

Subsequently, the reconstruction function 153 is executed to perform a reconstruction process on the projection data subjected to material decomposition and generate a reference material image (step S304).

Subsequently, the transformation function 155 is executed to separate the reference material image into a high-tube-voltage image x and a low-tube-voltage image y by image conversion such as grayscale conversion or color channel decomposition (step S306).

The high-tube-voltage image x is a reference material image reconstructed from projection data (e.g., 80 keV) in which the tube voltage of the X-ray tube 111 is greater than or equal to a threshold value and the low-tube-voltage image y is a reference material image reconstructed from projection data (e.g., 50 keV) in which the tube voltage of the X-ray tube 111 is less than the threshold value. The threshold value may be, for example, about 60 keV to 70 keV. The high-tube-voltage image x is an example of a “high-tube-voltage medical image” and the low-tube-voltage image y is an example of a “low-tube-voltage medical image.”

For example, when reference material images a and b are generated, the transformation function 155 is executed to separate the reference material image a into a high-tube-voltage image x and a low-tube-voltage image y and separate the reference material image b into a high-tube-voltage image x and a low-tube-voltage image y.

Subsequently, the denoising processing function 156 uses the trained model MDL to denoise each of the high-tube-voltage image x and the low-tube-voltage image y (step S308).

For example, the denoising processing function 156 is executed to read a third trained model MDLκ and a fourth trained model MDLλ defined by the model definition data stored in the memory 141, input the high-tube-voltage image x to the third trained model MDLκ, and input the low-tube-voltage image y to the fourth trained model MDLλ.

The third trained model MDLκ and the fourth trained model MDLλ, for example, may be implemented by a deep neural network such as a convolutional neural network. The third trained model MDLκ and the fourth trained model MDLλ may be implemented using other machine learning models such as a support vector machine, a decision tree, a random forest, and a logistic regression instead of a neural network.

The third trained model MDLκ and the fourth trained model MDLλ are trained to output an image in which noise has been reduced from an input image (i.e., a denoised image) in accordance with a certain input image.

Therefore, the denoising processing function 156 is executed to input the high-tube-voltage image x separated from the reference material image a to the third trained model MDLκ and therefore acquire a denoised image of the high-tube-voltage image x separated from the reference material image a (hereinafter referred to as a denoised high-tube-voltage image x) from the third trained model MDLκ.

Likewise, the denoising processing function 156 is executed to input the high-tube-voltage image x separated from the reference material image b to the third trained model MDLκ and therefore acquire a denoised high-tube-voltage image x, which is a denoised image of the high-tube-voltage image x separated from the reference material image b, from the third trained model MDLκ.

Furthermore, the denoising processing function 156 is executed to input the low-tube-voltage image y separated from the reference material image a to the fourth trained model MDLλ and therefore acquire a denoised image of the low-tube-voltage image y separated from the reference material image a (hereinafter referred to as a denoised low-tube-voltage image y) from the fourth trained model MDLλ.

Likewise, the denoising processing function 156 is executed to input the low-tube-voltage image y separated from the reference material image b to the fourth trained model MDLλ and therefore acquire the denoised low-tube-voltage image y, which is the denoised image of the low-tube-voltage image y separated from the reference material image b, from the fourth trained model MDLλ.

Subsequently, the transformation function 155 is executed to generate a denoised reference material image by coupling the denoised high-tube-voltage image x and the denoised low-tube-voltage image y for each reference material image in the inverse image conversion (inverse conversion such as grayscale conversion or color channel decomposition) (step S310).

For example, the transformation function 155 is executed to generate a denoised reference material image a′ by coupling the denoised high-tube-voltage image x and the denoised low-tube-voltage image y derived from the reference material image a by the inverse image conversion. Likewise, the transformation function 155 is executed to generate a denoised reference material image b′ by coupling the denoised high-tube-voltage image x and the denoised low-tube-voltage image y derived from the reference material image b by the inverse image conversion.

Subsequently, the transformation function 155 is executed to separate each of the denoised reference material image a′ and the denoised reference material image b′ into a signal image s and a noise image n by the whitening transform (step S312).

For example, the transformation function 155 is executed to separate the denoised reference material image a′ into a signal image s and a noise image n and separate the denoised reference material image b′ into a signal image s and a noise image n, by the whitening transform.

Subsequently, the denoising processing function 156 is executed to denoise each of the signal image s and the noise image n using the first trained model MDLα and the second trained model MDLβ (step S314).

The denoising processing function 156 is executed to input a signal image s separated from the denoised reference material image a′ to the first trained model MDLα and therefore acquire a denoised signal image s of the signal image s separated from the denoised reference material image a′ from the first trained model MDLα.

Likewise, the denoising processing function 156 is executed to input a signal image s separated from the denoised reference material image b′ to the first trained model MDLα and therefore acquire a denoised signal image s of the signal image s separated from the denoised reference material image b′ from the first trained model MDLα.

Furthermore, the denoising processing function 156 is executed to input the noise image n separated from the denoised reference material image a′ to the second trained model MDLβ and therefore acquire a denoised noise image n of the noise image n separated from the denoised reference material image a′ from the second trained model MDLβ.

Likewise, the denoising processing function 156 is executed to input a noise image n separated from the denoised reference material image b′ to the second trained model MDLβ and therefore acquire a denoised noise image n of the noise image n separated from the denoised reference material image b′ from the second trained model MDLβ.

Subsequently, the transformation function 155 is executed to generate a denoised reference material image (hereinafter referred to as a double-denoised reference material image) denoised by coupling the denoised signal image s and the denoised noise image n for each denoised reference material image by the inverse whitening transform (step S316). The double-denoised reference material image is an example of a “double-denoised medical image.”

For example, the transformation function 155 is executed to generate a double-denoised reference material image a″ by coupling a denoised signal image s and a denoised noise image n derived from the denoised reference material image a′ by the inverse whitening transform. Likewise, the transformation function 155 is executed to generate a double-denoised reference material image b″ by coupling a denoised signal image s and a denoised noise image n derived from the denoised reference material image b′ by the inverse whitening transform.

Subsequently, the output control function 157 is executed to output the double-denoised image (step S318). For example, the output control function 157 may cause the display 142 to display the double-denoised reference material image a″ and the double-denoised reference material image b″. Moreover, the output control function 157 may be executed to transmit the double-denoised reference material image a″ and the double-denoised reference material image b″ to an external device (e.g., a computer used by a doctor, engineer, or the like) via the communication interface 144. Thereby, the process of the present flowchart ends.

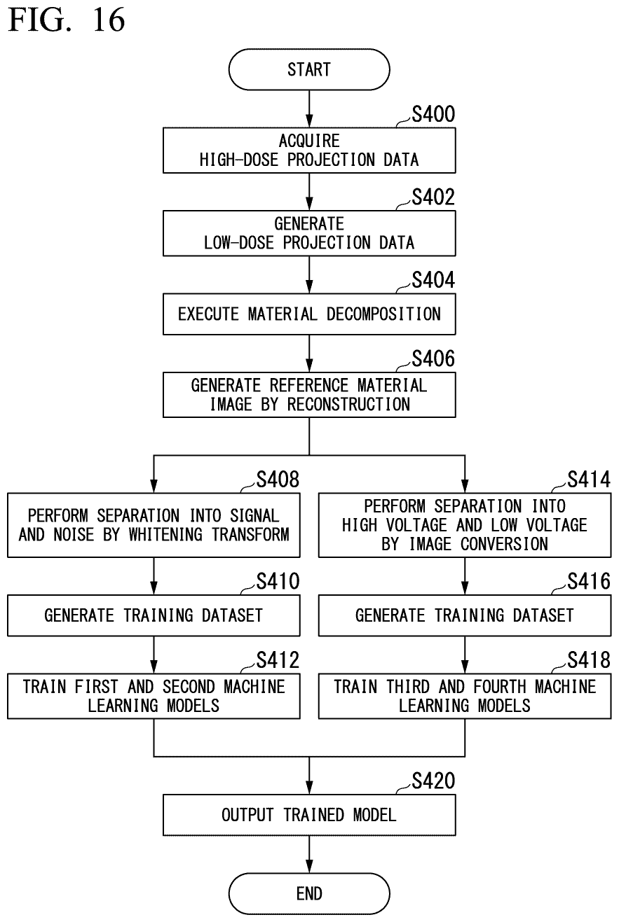

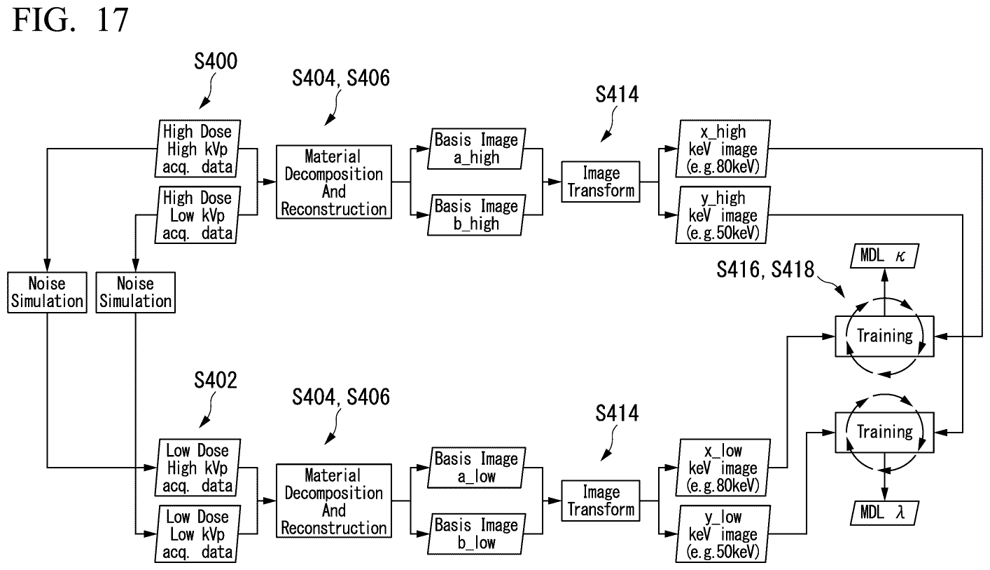

FIG. 16 is a flowchart showing a flow of a series of processing steps of the learning device 200 according to the fifth embodiment. FIG. 17 is a diagram schematically showing a flow of a series of processing steps of the learning device 200 according to the fifth embodiment. The process of the present flowchart is executed when the machine learning model MDL is trained.

First, the acquisition function 212 is executed to acquire projection data in which the X-ray dose is greater than or equal to a threshold value, i.e., high-dose projection data (step S400).

The acquisition function 212 is executed to acquire high-tube-voltage (high kVp) projection data and low-tube-voltage (low kVp) projection data as high-dose projection data.

Subsequently, the generation function 214 is executed to generate projection data with an X-ray dose below the threshold value, i.e., low-dose projection data, from the high-dose projection data acquired by the acquisition function 212 (step S402).

For example, the generation function 214 may be executed to generate low-dose, high-tube-voltage (high kVp) projection data from high-dose, high-tube-voltage (high kVp) projection data according to noise simulation and generate low-dose, low-tube-voltage (low kVp) projection data from high-dose, low-tube-voltage (low kVp) projection data.

Subsequently, the reconstruction function 216 is executed to perform material decomposition on the basis of a combination of high-dose, high-tube-voltage (high kVp) projection data and high-dose, low-tube-voltage (low kVp) projection data and perform material decomposition on the basis of a combination of low-dose, high-tube-voltage (high kVp) projection data and low-dose, low-tube-voltage (low kVp) projection data (step S404).

For example, the reconstruction function 216 is executed to use attenuation coefficients of two different types of reference materials a and b to discriminate between the reference materials a and b from a combination of high-dose, high-tube-voltage (high kVp) projection data and high-dose, low-tube-voltage (low kVp) projection data and to discriminate between the reference materials a and b from a combination of low-dose, high-tube-voltage (high kVp) projection data and low-dose, low-tube-voltage (low kVp) projection data.

Subsequently, the reconstruction function 216 is executed to perform a reconstruction process on the projection data subjected to the material decomposition and generate reference material images, which are CT images in which each reference material is enhanced or suppressed (step S406).

For example, the reconstruction function 216 is executed to perform a reconstruction process on high-dose projection data in which the reference materials a and b have been discriminated and generate a reference material image a high and a reference material image b_high. Likewise, the reconstruction function 216 is executed to perform a reconstruction process on low-dose projection data in which the reference materials a and b have been discriminated and generate a reference material image a_low and a reference material image b_low.

Subsequently, the transformation function 218 is executed to separate each of the four types of reference material images into a signal image s and a noise image n by the whitening transform (step S408).

For example, the transformation function 218 is executed to separate the reference material image b_high into a signal image s_high and a noise image n_high while separating the reference material image a_high into a signal image s_high and a noise image n_high by the whitening transform. Likewise, the transformation function 155 is executed to separate the reference material image b_low into a signal image s_low and a noise image n_low while separating the reference material image a low into a signal image s_low and a noise image n_low by the whitening transform.

Subsequently, the machine learning function 220 is executed to generate a first training dataset and a second training dataset (step S410).

For example, the machine learning function 220 is executed to generate a training dataset including only the datasets (i) and (ii) derived from the signal image s as a first training dataset and generate a training dataset including only the datasets (iii) and (iv) derived from the noise image n as a second training dataset.

Subsequently, the machine learning function 220 is executed to train a first trained model MDLα using the first training dataset and train a second trained model MDLβ using the second training dataset (step S412).

On the other hand, the transformation function 218 is executed to separate each of the four types of reference material images into a high-tube-voltage image x and a low-tube-voltage image y by image conversion (step S414).

For example, the transformation function 218 is executed to separate the reference material image b_high into a high-tube-voltage image x_high and a low-tube-voltage image y_high while separating the reference material image a_high into a high-tube-voltage image x_high and a low-tube-voltage image y_high by image conversion. Likewise, the transformation function 155 is executed to separate the reference material image b_low into a high-tube-voltage image x_low and a low-tube-voltage image y_low while separating the reference material image a_low into a high-tube-voltage image x low and a low-tube-voltage image y_low by image conversion.

Next, the machine learning function 220 is executed to generate a third training dataset and a fourth training dataset (step S416).

For example, the machine learning function 220 is executed to generate a dataset including only (v) and (vi) as the third training dataset and generate a dataset including only (vii) and (viii) as the fourth training dataset.
(v) A dataset in which a high-tube-voltage image x_high separated from a high-dose-derived reference material image a_high is associated as a target with a high-tube-voltage image x_low separated from a low-dose-derived reference material image a_low.
(vi) A dataset in which a high-tube-voltage image x_high separated from a high-dose-derived reference material image b_high is associated as a target with a high-tube-voltage image x_low separated from a low-dose-derived reference material image b_low.
(vii) A dataset in which a low-tube-voltage image y_high separated from a high-dose-derived reference material image a_high is associated as a target with a low-tube-voltage image y_low separated from a low-dose-derived reference material image a_low.
(viii) A dataset in which a low-tube-voltage image y_high separated from a high-dose-derived reference material image b_high is associated as a target with a low-tube-voltage image y_low separated from a low-dose-derived reference material image b_low.

Subsequently, the machine learning function 220 is executed to train a third trained model MDLκ using the third training dataset and train a fourth trained model MDLλ using the fourth training dataset (step S418).

For example, the machine learning function 220 is executed to input the high-tube-voltage image x_low of the third training dataset to the third trained model MDLκ.

The machine learning function 220 is executed to calculate a difference between the image output by the third trained model MDLκ in accordance with the input of the high-tube-voltage image x_low and the target high-tube-voltage image x_high. Also, the machine learning function 220 is executed to adjust parameters (a weighting coefficient and a bias component) of the third trained model MDLκ by performing error backpropagation using a stochastic gradient descent method or the like on the basis of the difference.

Furthermore, the machine learning function 220 is executed to input the low-tube-voltage image y_low of the fourth training dataset to the fourth trained model MDLλ. The machine learning function 220 is executed to calculate a difference between the image output by the fourth trained model MDLλ in accordance with the input of the low-tube-voltage image y_low and the target low-tube-voltage image y_high. Also, the machine learning function 220 is executed to adjust parameters (a weighting coefficient and a bias component) of the fourth trained model MDLλ by performing error backpropagation using a stochastic gradient descent method or the like on the basis of the difference.

Thus, the machine learning function 220 is executed to iterate the above-described process until the number of training iterations reaches a specified number and store model definition data defining the first trained model MDLα, the second trained model MDLβ, the third trained model MDLκ, and the fourth trained model MDLλ in the memory 208 when the number of iterations reaches the specified number.

Subsequently, the output control function 222 is executed to transmit model definition data defining the first trained model MDLα, the second trained model MDLβ, the third trained model MDLκ, and the fourth trained model MDLλ trained by the machine learning function 220 to each X-ray CT device 100 via the communication interface 202 (step S420). Thereby, the process of the present flowchart ends.

According to the above-described fifth embodiment, not only the signal image s and/or the noise image n are denoised, but also the reference material image is denoised. Thus, it is possible to further improve the accuracy of noise reduction in spectral imaging by performing denoising twice.

### Sixth Embodiment

Hereinafter, a sixth embodiment will be described. The sixth embodiment is different from the first to fifth embodiments in that both the high-tube-voltage image x and the low-tube-voltage image y are denoised using either the third trained model MDLκ or the fourth trained model MDLλ. Hereinafter, the differences from the first to fifth embodiments will be mainly described and the description of portions identical to those in the first to fifth embodiments will be omitted. In addition, in the description of the sixth embodiment, parts identical to those in the first to fifth embodiments are denoted by the same reference signs.

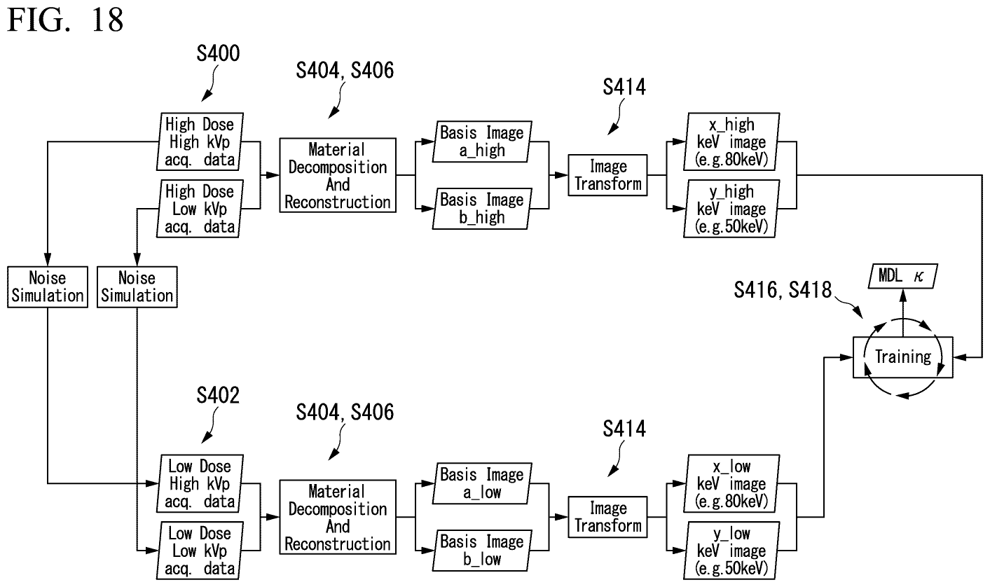

FIG. 18 is a diagram schematically showing a flow of a series of processing steps of the learning device 200 according to the sixth embodiment.

The machine learning function 220 according to the sixth embodiment is executed to generate a third training dataset for training the third trained model MDLκ as the processing of step S416. For example, the machine learning function 220 is executed to generate a dataset including the above-described (v) to (viii) as the third training dataset.

The machine learning function 220 is executed to train a third trained model MDLκ using the third training dataset including (v) to (viii) as the processing of step S418.

According to the above-described sixth embodiment, the X-ray CT device 100 separates each of the reference material image a and the reference material image b into a high-tube-voltage image x and a low-tube-voltage image y by image conversion. The X-ray CT device 100 inputs the high-tube-voltage image x and the low-tube-voltage image y to the third trained model MDLκ trained on the basis of the third training dataset and acquires the denoised high-tube-voltage image x and the denoised low-tube-voltage image y from the third trained model MDLκ.

In the above-described fifth embodiment, the third training dataset includes only the datasets (v) and (vi) derived from the high-tube-voltage image x. In contrast, in the sixth embodiment, the third training dataset includes the datasets (vii) and (viii) derived from the low-tube-voltage image y in addition to the datasets (v) and (vi) derived from the high-tube-voltage image x. By solely using the third trained model MDLκ trained using such a third training dataset it is possible to denoise the low-tube-voltage image y as well as the high-tube-voltage image x.

Although a case where the third trained model MDLκ is solely used to denoise both the high-tube-voltage image x and the low-tube-voltage image y has been described in the above-described third embodiment, the present invention is not limited to the above. For example, the fourth trained model MDLλ may be solely used to denoise both the high-tube-voltage image x and the low-tube-voltage image y. In this case, a fourth training dataset including (v) to (viii) is used to train the fourth trained model MDLλ.

### Seventh Embodiment

Hereinafter, a seventh embodiment will be described. The seventh embodiment is different from the first to sixth embodiments in that either the high-tube-voltage image x or the low-tube-voltage image y is denoised using either the third trained model MDLκ or the fourth trained model MDLλ. Hereinafter, the differences from the first to sixth embodiments will be mainly described and the description of portions identical to those in the first to sixth embodiments will be omitted. In addition, in the description of the seventh embodiment, parts identical to those in the first to sixth embodiments are denoted by the same reference signs.

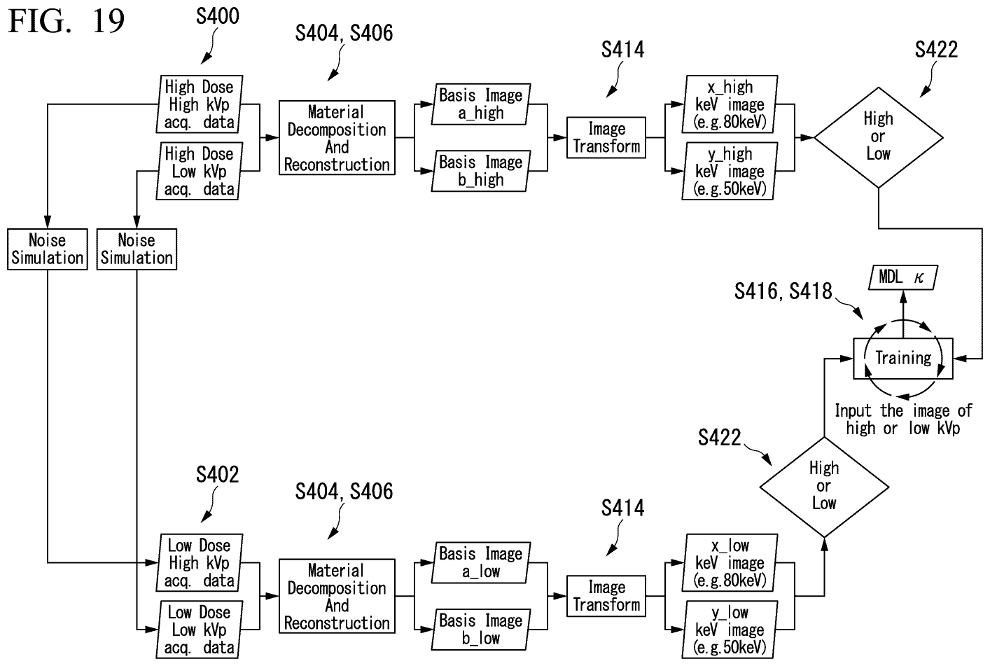

FIG. 19 is a diagram schematically showing a flow of a series of processing steps of the learning device 200 according to the seventh embodiment.

In the machine learning function 220 according to the seventh embodiment, when the transformation function 218 is executed to separate each of the four types of reference material images into a high-tube-voltage image x and a low-tube-voltage image y by image conversion as the processing of S414, it is determined whether the denoise request image is the high-tube-voltage image x or the low-tube-voltage image y (step S422).

In the seventh embodiment, it is assumed that the denoising request image is a high-tube-voltage image x.

When the denoising request image is a high-tube-voltage image x, the machine learning function 220 is executed to extract only high-tube-voltage images x from among a plurality of high-tube-voltage images x and a plurality of low-tube-voltage images y and generate a third training dataset including only the high-tube-voltage images x as the processing of S416. For example, the machine learning function 220 is executed to generate a third training dataset including the above-described (v) and (vi).

As the processing of step S418, the machine learning function 220 is executed to train a third trained model MDLκ using the third training dataset including (v) and (vi).

According to the above-described seventh embodiment, the X-ray CT device 100 separates each of the reference material image a and the reference material image b into a high-tube-voltage image x and a low-tube-voltage image y by image conversion. The X-ray CT device 100 inputs the high-tube-voltage image x to the third trained model MDLκ trained on the basis of the third training dataset and acquires the denoised high-tube-voltage image x from the third trained model MDLκ. Also, the X-ray CT device 100 generates a denoised reference material image by coupling the denoised high-tube-voltage image x and the low-tube-voltage image y by the inverse image transform. Thus, it is possible to generate a denoised reference material image by denoising only one of the high-tube-voltage image x and the low-tube-voltage image y without denoising both of them.

Although a case where the third trained model MDLκ is solely used to denoise the high-tube-voltage image x has been described in the above-described seventh embodiment, the present invention is not limited thereto. For example, the fourth trained model MDLλ may be solely used to denoise the high-tube-voltage image x.

### Other Embodiments

Hereinafter, other embodiments will be described. Although a case where the processing circuitry 150 of the console device 140 of the X-ray CT device 100 includes the system control function 151, the preprocessing function 152, the reconstruction function 153, the image processing function 154, the transformation function 155, the denoising processing function 156, and the output control function 157 has been described in the above-described embodiment, the present invention is not limited thereto. For example, particularly, some or all of the preprocessing function 152, the reconstruction function 153, the image processing function 154, the transformation function 155, and the denoising processing function 156 among these multiple functions, may be provided in the learning device 200 or another external device.

Although a case where the processing circuitry 210 of the learning device 200 includes the acquisition function 212, the generation function 214, the reconstruction function 216, the transformation function 218, the machine learning function 220, and the output control function 222 has been described, the present invention is not limited thereto. For example, particularly, some or all of the generation function 214, the reconstruction function 216, the transformation function 218, and the machine learning function 220 among these multiple functions may be provided in the processing circuitry 150 of the X-ray CT device 100.

Moreover, it is possible to use a machine learning model MDL trained on projection data acquired by a different acquisition method. For example, a single machine learning model MDL trained on a monochromatic 70 keV image created from projection data acquired by a DECT device may be applied to three reference material images of iodine, water, and bone generated by a PCCT device.

Moreover, it is possible to use a machine learning model MDL trained on projection data acquired by a different detector. For example, a machine learning model MDL trained on a counting image generated from projection data acquired by an energy integral detector (EID) may be applied to a counting image generated from projection data acquired by a PCCT device.

While certain embodiments of the present invention have been described, these embodiments have been presented by way of example only, and are not intended to limit the scope of the inventions. These embodiments may be embodied in a variety of other forms. Various omissions, substitutions, and modifications may be made without departing from the spirit of the inventions. The inventions described in the accompanying claims and their equivalents are intended to cover such embodiments or modified examples as would fall within the scope and spirit of the inventions.

What is claimed is:

1. A medical information processing method comprising:
acquiring projection data of X-rays radiated to a subject;
generating a medical image from the projection data by a reconstruction process;
separating the medical image into a signal image in which the number of signal components is more than the number of noise components and a noise image in which the number of noise components is more than the number of signal components by a whitening transform;
denoising at least one of the signal image and the noise image using a first trained model that is a machine learning model trained on the basis of a training dataset in which a first output image having less noise than a first input image is associated as a target with the first input image;
generating a denoised medical image that is the medical image that has been denoised by coupling the signal image that has been denoised and the noise image that has been denoised by an inverse whitening transform when both the signal image and the noise image have been denoised; and
generating the denoised medical image by coupling one image that has been denoised between the signal image and the noise image and the other image that has not been denoised by the inverse whitening transform when one of the signal image and the noise image has been denoised.

2. The medical information processing method according to claim 1, further comprising:
generating a plurality of different types of medical images on the basis of attenuation coefficients of a plurality of predetermined reference materials; and
separating each of the plurality of medical images into the signal image and the noise image by the whitening transform.

3. The medical information processing method according to claim 1, wherein the plurality of medical images include at least one of a reference material image, a virtual monochromatic X-ray image, a virtual plain image, an iodine map, an effective atomic number image, an electron density image, an image reconstructed for each energy bin in spectral imaging, an image reconstructed under a normal resolution mode that is a mode in which a resolution is less than or equal to a threshold value, and an image reconstructed under a super-resolution mode that is a mode in which the resolution exceeds the threshold value.

4. The medical information processing method according to claim 1, further comprising:
separating the medical image into a high-tube-voltage medical image that is the medical image reconstructed from the projection data in which a tube voltage as a voltage of an X-ray tube for generating the X-rays is greater than or equal to a threshold value and a low-tube-voltage medical image that is the medical image reconstructed from the projection data in which the tube voltage is less than the threshold value by an image transform;
denoising at least one of the high-tube-voltage medical image and the low-tube-voltage medical image using a second trained model that is a machine learning model trained on the basis of a training dataset in which a second output image having less noise than a second input image is associated as a target with the second input image;
generating the denoised medical image by coupling the high-tube-voltage medical image that has been denoised and the low-tube-voltage medical image that has been denoised by an inverse image transform when both the high-tube-voltage medical image and the low-tube-voltage medical image have been denoised; and
generating the denoised medical image by coupling one image that has been denoised between the high-tube-voltage medical image and the low-tube-voltage medical image and the other image that has not been denoised by the inverse image transform when one of the high-tube-voltage medical image and the low-tube-voltage medical image has been denoised.

5. The medical information processing method according to claim 4, further comprising:
separating the denoised medical image into the signal image and the noise image by the whitening transform;
denoising at least one of the signal image and the noise image using the first trained model;
generating a double-denoised medical image as the denoised medical image that has been denoised by coupling the signal image that has been denoised and the noise image that has been denoised by the inverse whitening transform when both the signal image and the noise image have been denoised; and
generating the double-denoised medical image by coupling one image that has been denoised between the signal image and the noise image and the other image that has not been denoised by the inverse whitening transform when one of the signal image and the noise image has been denoised.

6. A medical information processing device comprising:
processing circuitry configured to
acquire projection data of X-rays radiated to a subject;
generate a medical image from the projection data by a reconstruction process;
separate the medical image into a signal image in which the number of signal components is more than the number of noise components and a noise image in which the number of noise components is more than the number of signal components by a whitening transform; and
denoise at least one of the signal image and the noise image using a first trained model that is a machine learning model trained on the basis of a training dataset in which a first output image having less noise than a first input image is associated as a target with the first input image,
wherein the processing circuitry
generates a denoised medical image that is the medical image that has been denoised by coupling the signal image that has been denoised and the noise image that has been denoised by an inverse whitening transform when both the signal image and the noise image have been denoised, and
generating the denoised medical image by coupling one image that has been denoised between the signal image and the noise image and the other image that has not been denoised by the inverse whitening transform when one of the signal image and the noise image has been denoised.

7. A computer-readable non-transitory storage medium storing a program for causing a computer to:
acquire projection data of X-rays radiated to a subject;
generate a medical image from the projection data by a reconstruction process;
separate the medical image into a signal image in which the number of signal components is more than the number of noise components and a noise image in which the number of noise components is more than the number of signal components by a whitening transform;
denoise at least one of the signal image and the noise image using a first trained model that is a machine learning model trained on the basis of a training dataset in which a first output image having less noise than a first input image is associated as a target with the first input image;
generate a denoised medical image that is the medical image that has been denoised by coupling the signal image that has been denoised and the noise image that has been denoised by an inverse whitening transform when both the signal image and the noise image have been denoised; and
generate the denoised medical image by coupling one image that has been denoised between the signal image and the noise image and the other image that has not been denoised by the inverse whitening transform when one of the signal image and the noise image has been denoised.
